(19) United States
(12) Patent Application Publication
(10) Pub. No.: US 2012/0052020 A1
(43) Pub. Date: Mar. 1, 2012

(54) POLYMER COMBINATION FROM AT LEAST TWO DIFFERENT N-VINYLPYRROLIDONE/N-VINYLCAPROLACTAM COPOLYMERS FOR COSMETIC HAIR SHINE PRODUCTS

(76) Inventors: SUSANNE SCHMARJE, Hamburg (DE); Uwe Bergemann, Hamburg (DE)

(21) Appl. No.: 13/291,434

(22) Filed: Nov. 8, 2011

Related U.S. Application Data
(63) Continuation of application No. PCT/EP2010/054679, filed on Apr. 9, 2010.

(30) Foreign Application Priority Data
May 8, 2009 (DE) 10 2009 020 552.7

(51) Int. Cl. A61K 8/81; A61K 8/02; A61Q 5/06

(52) U.S. Cl. 424/47; 424/70.15

(57) ABSTRACT

Products for treating keratin-containing fibers containing, in a cosmetically acceptable carrier, (i) 1.0% by weight to 2.8% by weight of at least one copolymer (a) having at least one structural unit of formula (I) and at least one structural unit of formula (II) and at least one structural unit of formula (III), wherein R¹ is hydrogen or a methyl group, X¹ is oxygen or an NH group, and R² and R³ independently represent a (C₁ to C₄) alkyl group, and (ii) 0.4% by weight to 1.4% by weight of at least one copolymer (b) having at least one structural unit of formula (I) and at least one structural unit of formula (II) and at least one structural unit of formula (IV), wherein R⁴ is hydrogen or a methyl group, X² is oxygen or an NH group, and R⁵ and R⁶ independently represent a (C₁ to C₄) alkyl group.

### CROSS REFERENCE TO RELATED APPLICATIONS

This application is a continuation of International Patent Application No. PCT/EP2010/054679 filed 9 Apr. 2010, which claims priority to German Patent Application No. 10 2009 020 552.7 filed 8 May 2009, both of which are incorporated herein by reference.

The present invention relates to agents for hair treatment containing a specific mixture of a polymer combination made up of at least two different N-vinylpyrrolidone/N-vinylcaprolactam copolymers; use of the agents for the temporary deformation and/or care of keratin-containing fibers; and aerosol sprays or aerosol foams based on those agents.

“Keratin-containing fibers” are understood to generally include all animal hairs (e.g., wool, horsehair, angora hair, furs, feathers, and products or textiles fabricated therefrom). The keratinic fibers are, however, preferably human hair.

An attractive-looking hairstyle is generally regarded these days as an indispensable element of a well-groomed appearance. Considering fashion trends, more and more hairstyles regarded as chic are ones that, for many types of hair, can be constructed or maintained for a longer period of time of up to several days, using only setting ingredients. Hair treatment agents that provide permanent or temporary shaping of the hair therefore play an important role. Temporary shaping actions that are intended to yield good hold without impairing the hair's healthy appearance such as its shine can be achieved, for example, using hair sprays, hair waxes, hair gels, hair foams, blow-dry waves, etc.

Corresponding agents for temporary shaping usually contain synthetic polymers as a shaping component. Preparations that contain a dissolved or dispersed polymer can be applied onto the hair by propellant gases or by a pump mechanism. Hair gels and hair waxes in particular, however, are generally not applied directly onto the hair but rather distributed in the hair using a comb or the hands.

An initially important property of an agent for temporary deformation of keratinic fibers, hereinafter also called a “styling agent,” is to impart the strongest possible hold to the treated fibers in the shape that is generated. If the keratinic fibers involved are human hairs, terms also used are a strong “hairstyle hold” or a high “degree of hold” of the styling agent. Hairstyle hold is determined substantially by the nature and quantity of the synthetic polymer used, although other ingredients of the styling agent can also have an influence.

In addition to a high level of hold, styling agents must also meet a large number of further requirements. These can be subdivided roughly into hair properties; formulation properties (e.g. properties of the foam, gel, or sprayed aerosol); and properties that relate to the handling of the styling agent, with the properties on the hair being of particular importance. These include moisture resistance, low tack, and a balanced conditioning effect in particular. In addition, if possible, a styling agent should be universally usable for all types of hair.

A plurality of synthetic polymers utilized in styling agents has been developed to meet various requirements. The polymers can be subdivided into cationic, anionic, nonionic, and amphoteric film-forming and/or setting polymers. Ideally, even when a small quantity is applied to the hair, the polymers yield a polymer film that imparts a strong hold to the hairstyle while is sufficiently flexible not to break under stress. If the polymer is too fragile, this results in the formation of “film plaques” (i.e., residues that detach as the hair moves and gives the impression that the user of the corresponding styling agent has dandruff).

The temporarily styled hair should moreover, in addition to the strong hairstyle hold, look healthy and natural. Hair shine plays a dominant role here. Shine agents are therefore often added in sufficient quantity to the hair styling agents. These shine agents include oils or shine-imparting pigments such as mica particles. Shine-imparting particles have the disadvantage that they detach from the hair over time and can collect on, for example, clothing or facial skin. Oils make the hair heavy and in some cases result in degraded adhesion of the film-forming or setting polymers on the hair. This can result in the constructed hairstyle not being immobilized for a sufficient period of time by the film-forming or setting polymers (i.e., the hairstyle collapses more quickly).

The present invention therefore attempts to make available an agent for temporary deformation and/or care of keratinic fibers having a high degree of hold and producing outstanding shine on the keratin-containing fibers.

It has now been surprisingly found that this can be achieved by combination of specific polymers.

A first subject of the invention is therefore an agent for treating keratin-containing fibers, particularly human hair, containing in a cosmetically acceptable carrier:
i) 1.0 wt % to 2.8 wt %, preferably 1.5 wt % to 2.8 wt %, based on total weight of the cosmetic agent, of at least one copolymer (a) having at least one structural unit of formula (I), at least one structural unit of formula (II) and at least one structural unit of formula (III)

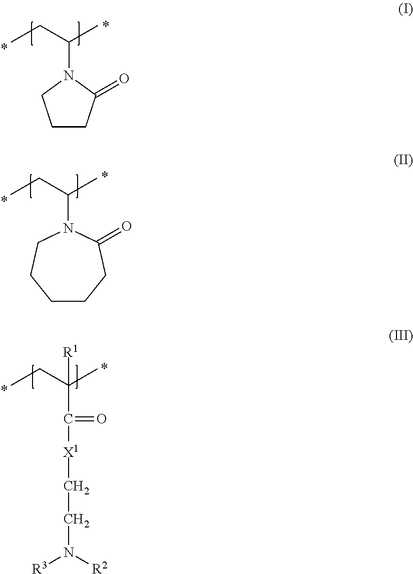

wherein

R¹ is a hydrogen atom or a methyl group,

X¹ is an oxygen atom or an NH group,

R² and R³, mutually independently, are a (C₁ to C₄) alkyl group, and
ii) 0.4 wt % to 1.4 wt %, preferably 0.6 wt % to 1.3 wt %, based on total weight of the cosmetic agent, of at least one copolymer (b) having at least one structural unit of formula (I), at least one structural unit of formula (II) and at least one structural unit of formula (IV)

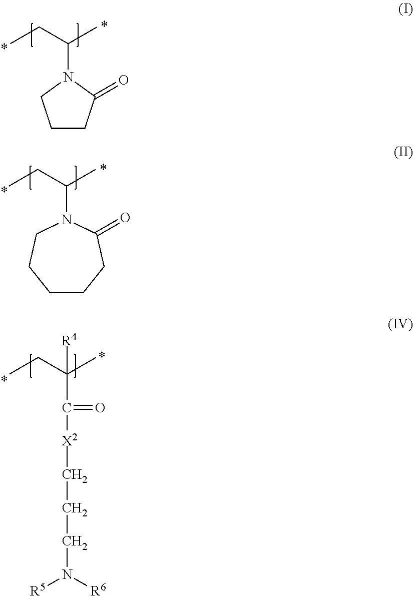

wherein

R⁴ is a hydrogen atom or a methyl group,

X² is an oxygen atom or an NH group,

R⁵ and R⁶, mutually independently, are a (C₁ to C₄) alkyl group.

All quantity range indications for purposes of the invention are understood to incorporate the respectively recited upper and lower limits.

According to the above formulae and all subsequent formulae, a chemical bond identified by the symbol “*” represents a free valence of the corresponding structural fragment.

Examples of (C₁ to C₄) alkyl groups according to formulae (III) and (IV) are methyl, ethyl, isopropyl, n-propyl, n-butyl, sec-butyl, isobutyl, and tert-butyl.

Preferred agents according to the present invention contain at least one such copolymer (a) that, in accordance with formula (III), conforms to at least one of the following parameters (preferably all three):

R¹ is a methyl group,

X¹ is an oxygen atom,

R² and R³ are a methyl group.

Very particularly preferably, the agent contains as copolymer (a) a terpolymer of N-vinylpyrrolidone, N-vinylcaprolactam, and N,N-dimethylaminoethyl methacrylate. Such copolymers can be procured, for example, under the trade name Advantage LC-E (INCI name: Vinylcaprolactam/VP/Dimethylaminoethyl Methacrylate Copolymer, Laurylpyrrolidone; 37 wt % active substance in ethanol with added N-laurylpyrrolidone) or Advantage LC-A (INCI name: Vinylcaprolactam/VP/Dimethylaminoethyl Methacrylate Copolymer; 37 wt % active substance in ethanol) from the ISP company.

Preferred agents according to the present invention contain at least one copolymer (b) that, in accordance with formula (IV), conforms to at least one of the following parameters (particularly preferably all three together):

R⁴ is a methyl group,

X² is an NH group,

R⁵ and R⁶ are a methyl group.

Very particularly preferably, the agent contains as copolymer (b) a terpolymer of N-vinylpyrrolidone, N-vinylcaprolactam, and N,N-dimethylaminopropyl methacrylamide. Such copolymers are obtainable, for example, under the trade name Aquaflex SF 40 (INCI name: VP/Vinyl Caprolactam/DMAPA Acrylates Copolymer, Alcohol Denat.; 38 to 42 wt % active substance in ethanol) from the ISP company.

Furthermore, a mixing ratio of copolymer (a) and copolymer (b) in a weight ratio range (i) to (ii) from 1 to 1 to 1 to 5, particularly from 1 to 1.5 to 1 to 2.5, is suitable for achieving maximum hold with ideal hair shine. This mixing ratio is preferred for all embodiments of copolymers (a) and (b). This is the case even when at least one of the subsequent preferred additional ingredients is added.

A particularly preferred agent for treating keratin-containing fibers, particularly human hair, contains in a cosmetically acceptable carrier:
i) 1.0 wt % to 2.8 wt %, preferably 1.5 wt % to 2.8 wt %, based on total weight of the cosmetic agent, of at least one copolymer (a) having at least one structural unit of formula (I), at least one structural unit of formula (II) and at least one structural unit of formula (III-1)

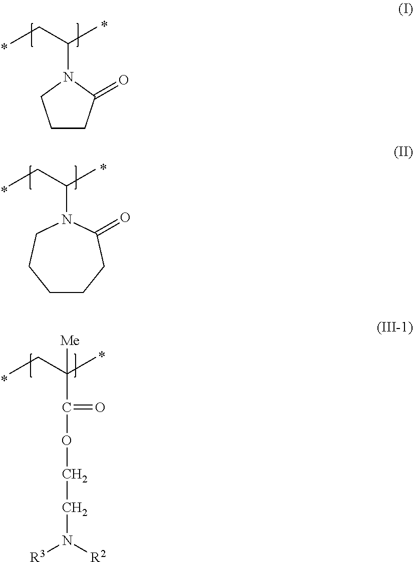

wherein

R² and R³, mutually independently, are a (C₁ to C₄) alkyl group, and
ii) 0.4 wt % to 1.4 wt %, preferably 0.6 wt % to 1.3 wt %, based on total weight of the cosmetic agent, of at least one copolymer (b) having at least one structural unit of formula (I), at least one structural unit of formula (II) and at least one structural unit of formula (IV-1)

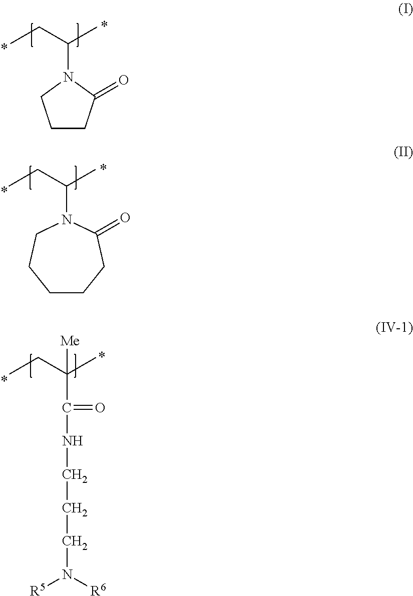

wherein

R⁵ and R⁶, mutually independently, are a (C₁ to C₄) alkyl group.

Examples of (C₁ to C₄) alkyl groups according to formulae (III-1) and (IV-1) are methyl, ethyl, isopropyl, n-propyl, n-butyl, sec-butyl, isobutyl, and tert-butyl.

Very particularly preferred agents according to the present invention for treating keratin-containing fibers, in particular human hair, contain in a cosmetically acceptable carrier:
i) 1.0 wt % to 2.8 wt %, preferably 1.5 wt % to 2.8 wt %, based on total weight of the cosmetic agent, of at least one terpolymer of N-vinylpyrrolidone, N-vinylcaprolactam, and N,N-dimethylaminoethyl methacrylate as copolymer (a), and
ii) 0.4 wt % to 1.4 wt %, preferably 0.6 wt % to 1.3 wt %, based on total weight of the cosmetic agent, of at least one terpolymer of N-vinylpyrrolidone, N-vinylcaprolactam, and N,N-dimethylaminopropyl methacrylamide as copolymer (b).

A particularly preferred agent for treating keratin-containing fibers, in particular human hair, contains in a cosmetically acceptable carrier:
i) 1.0 wt % to 2.8 wt %, preferably 1.5 wt % to 2.8 wt %, based on total weight of the cosmetic agent, of at least one copolymer (a) having at least one structural unit of formula (I), at least one structural unit of formula (II) and at least one structural unit of formula (III-1)

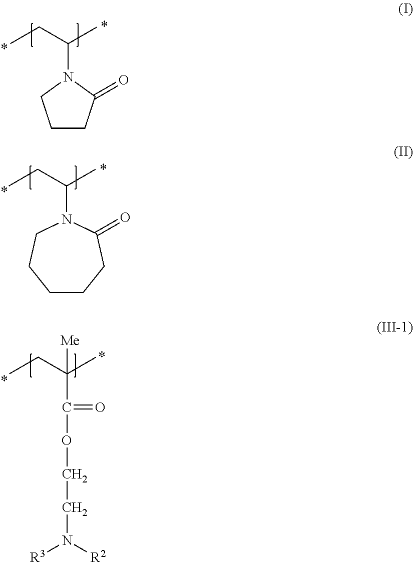

wherein

R² and R³, mutually independently, are a (C₁ to C₄) alkyl group,
ii) 0.4 wt % to 1.4 wt %, preferably 0.6 wt % to 1.3 wt %, based on total weight of the cosmetic agent, of at least one copolymer (b) having at least one structural unit of formula (I), at least one structural unit of formula (II) and at least one structural unit of formula (IV-1)

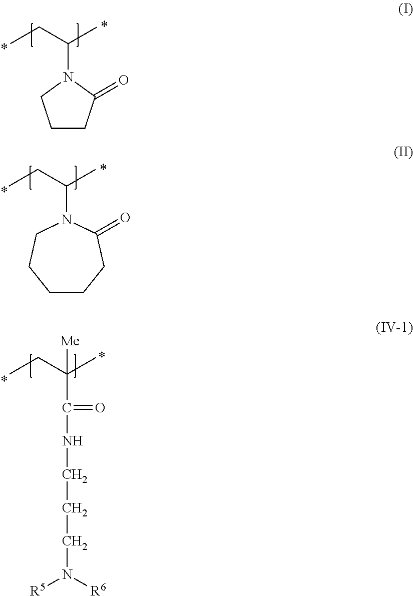

wherein

R⁵ and R⁶, mutually independently, are a (C₁ to C₄) alkyl group,

wherein copolymer (a) and copolymer (b) are present in a weight ratio range (i) to (ii) from 1 to 1 to 1 to 5, particularly from 1 to 1.5 to 1 to 2.5.

Examples of (C₁ to C₄) alkyl groups according to formulae (III-1) and (IV-1) are methyl, ethyl, isopropyl, n-propyl, n-butyl, sec-butyl, isobutyl, and tert-butyl.

Very particularly preferred agents for treating keratin-containing fibers, particularly human hair, contain in a cosmetically acceptable carrier:
i) 1.0 wt % to 2.8 wt %, preferably 1.5 wt % to 2.8 wt %, based on total weight of the cosmetic agent, of at least one terpolymer of N-vinylpyrrolidone, N-vinylcaprolactam, and N,N-dimethylaminoethyl methacrylate as copolymer (a), and
ii) 0.4 wt % to 1.4 wt %, preferably 0.6 wt % to 1.3 wt %, based on total weight of the cosmetic agent, of at least one terpolymer of N-vinylpyrrolidone, N-vinylcaprolactam, and N,N-dimethylaminopropyl methacrylamide as copolymer (b),
   wherein copolymer (a) and copolymer (b) are present in a weight ratio range (i) to (ii) from 1 to 1 to 1 to 5, particularly from 1 to 1.5 to 1 to 2.5.

Agents according to the present invention contain the ingredients or active substances in a cosmetically acceptable carrier.

Preferred cosmetically acceptable carriers are aqueous, alcoholic, or aqueous alcoholic media preferably having at least 10 wt % water, based on total agent. The alcohols contained can be, in particular, lower alcohols having 1 to 4 carbon atoms and usually used for cosmetic purposes (e.g., ethanol and isopropanol).

In a preferred embodiment, the agent additionally contains at least one alcohol having 2 to 6 carbon atoms and 1 to 3 hydroxyl groups. This additional alcohol is preferably chosen from ethanol, ethylene glycol, isopropanol, 1,2-propylene glycol, 1,3-propylene glycol, glycerol, n-butanol, and/or 1,3-butylene glycol. A very particularly preferred alcohol is ethanol.

The additional alcohol having 2 to 6 carbon atoms and 1 to 3 hydroxyl groups is present in the agent (particularly in the presence of at least one propellant) preferably in an amount from 40 wt % to 65 wt %, particularly 40 wt % to 50 wt %, based on total weight of the cosmetic agent.

Organic solvents or mixture of solvents having a boiling point under 400° C. can be present as additional co-solvents in an amount from 0.1 to 15 weight percent, preferably 1 to 10 weight percent, based on total agent. Unbranched or branched hydrocarbons such as pentane, hexane, isopentane, and cyclic hydrocarbons such as cyclopentane and cyclohexane, are particularly suitable as additional co-solvents. Further particularly preferred water-soluble solvents are polyethylene glycol and propylene glycol, in an amount of up to 30 wt % based on total agent.

The addition of propylene glycol and/or polyethylene glycol and/or polypropylene glycol increases the flexibility of the polymer film formed when the agent according to the present invention is used. If a flexible hold is desired, the agents therefore preferably contain 0.01 to 30 wt % polyethylene glycol and/or polypropylene glycol, based on total agent.

The agents preferably have a pH from 2 to 11. More preferably, the pH range is from 2 to 8. References to pH here, for purposes of this document, to pH at 25° C. unless otherwise noted.

It has proven suitable according to the present invention if the agent optionally additionally contains at least one N—(C₆ to C₂₀) alkylpyrrolidone. N-laurylpyrrolidone is a suitable N—(C₆ to C₂₀) alkylpyrrolidone preferred according to the present invention.

It has been possible to increase the effects according to the present invention by adding at least one (C₂ to C₆) trialkyl citrate to the agent. It is therefore preferred if the agents additionally contain at least one compound of formula (E)

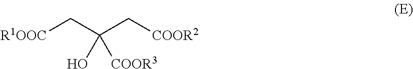

wherein R¹, R², and R³, mutually independently, are a (C₂ to C₆) alkyl group.

Examples of a (C₂ to C₆) alkyl group according to formula (E) are methyl, ethyl, isopropyl, n-propyl, n-butyl, sec-butyl, isobutyl, tert-butyl, n-pentyl, neopentyl, isopentyl, n-hexyl.

Triethyl citrate stands out as a particularly preferred compound of formula (E).

Agents according to the present invention contain compounds of formula (E) preferably in an amount from 0.01 to 1 wt %, particularly 0.05 to 0.3 wt %, based on total weight of the agent.

It has been possible to achieve a similar increase in the effects according to the present invention by adding isopropyl myristate. Isopropyl myristate is present in agents according to the present invention preferably in a quantity from 0.01 to 1 wt %, particularly 0.05 to 0.3 wt %, based on total weight of the agent.

To intensify the effect according to the present invention, the agents preferably also contain at least one surfactant. Nonionic, anionic, cationic, and ampholytic surfactants are suitable in principle. Ampholytic or amphoteric surfactants include zwitterionic surfactants and ampholytes. The surfactants can already have an emulsifying effect. Use of at least one nonionic surfactant and/or at least one cationic surfactant is preferred in the context of this embodiment of the invention.

The additional surfactants are present in the agent preferably in a quantity from 0.01 wt % to 5 wt %, more preferably 0.05 wt % to 0.5 wt %, based on total weight of the agent.

It is particularly preferred if the agents additionally contain at least one nonionic surfactant.

Nonionic surfactants contain a hydrophilic group such as a polyol group, a polyalkylene glycol ether group, or a combination of a polyol and polyglycol ether group. Such compounds include:
addition products of 2 to 100 mol ethylene oxide and/or 1 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group,
addition products, end-capped with a methyl or C₂ to C₆ alkyl group, of 2 to 50 mol ethylene oxide and/or 1 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group, such as the grades obtainable under the marketing designations Dehydrol® LS, Dehydrol® LT (Cognis),
C₁₂ to C₃₀ fatty acid mono- and diesters of addition products of 1 to 30 mol ethylene oxide with glycerol,
addition products of 5 to 60 mol ethylene oxide with castor oil and hardened castor oil,
polyol fatty acid esters such as the commercial product Hydagen® HSP (Cognis), or Sovermol® grades (Cognis),
alkoxylated triglycerides,
alkoxylated fatty acid alkyl esters of formula (T-I)

R¹CO—(OCH₂CHR²)_{w}OR² (T-I),

wherein R¹CO is a linear or branched, saturated and/or unsaturated acyl residue having 6 to 22 carbon atoms, R² is hydrogen or methyl, R³ is linear or branched alkyl residues having 1 to 4 carbon atoms, and w is a number from 1 to 20,
amine oxides,
hydroxy mixed ethers such as those described in German Application 19738866,
sorbitan fatty acid esters and addition products of ethylene oxide with sorbitan fatty acid esters, for example, polysorbates,
sugar fatty acid esters and addition products of ethylene oxide with sugar fatty acid esters,
addition products of ethylene oxide with fatty acid alkanolamides and fatty amines,
sugar surfactants of the alkyl and alkenyl oligoglycoside types, according to formula (T-II)

R⁴O-[G]ₚ (T-II),

wherein R⁴ is an alkyl or alkenyl residue having 4 to 22 carbon atoms, G is a sugar residue having 5 or 6 carbon atoms, and p is a number from 1 to 10. They can be obtained by relevant methods of preparative organic chemistry.
The alkyl and alkenyl oligoglycosides can be derived from aldoses or ketoses having 5 or 6 carbon atoms, preferably from glucose. Preferred alkyl or alkenyl oligoglycosides are alkyl and/or alkenyl oligoglucosides. The index number p in general formula (T-II) indicates the degree of oligomerization (DP) (i.e., the distribution of mono- and oligoglycosides) and is a number from 1 to 10. Whereas p in the individual molecule must always be integral, and here can assume especially the values p=1 to 6, the value p for a specific alkyl oligoglycoside is an analytically ascertained calculated value that usually represents a fractional number. Alkyl and/or alkenyl oligoglycosides having an average degree of oligomerization p from 1.1 to 3.0 are preferably used. In terms of applications engineering, those alkyl and/or alkenyl oligoglycosides whose degree of oligomerization is less than 1.7, and particularly from 1.2 to 1.4, are preferred. The alkyl or alkenyl residue R⁴ can be derived from primary alcohols having 4 to 11, preferably 8 to 10 carbon atoms. Typical examples are butanol, hexanol, octanol, decanol, and undecyl alcohol as well as industrial mixtures thereof, such as those obtained upon hydrogenation of industrial fatty acid methyl esters or in the course of the hydrogenation of aldehydes from Roelen oxosynthesis. Preferred are alkyl oligoglucosides of chain length C₈ to C₁₀ (DP=1 to 3), which occur as the first runnings upon distillational separation of industrial C₈ to C₁₈ coconut oil alcohol and can be contaminated with a proportion of less than 6 wt % C₁₋₂ alcohol, and alkyl oligoglucosides based on industrial C_{9/11} oxoalcohols (DP=1 to 3). The alkyl or alkenyl residue R¹⁵ can furthermore also be derived from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and industrial mixtures thereof, which can be obtained as described above. Alkyl oligoglucosides based on hardened C_{12/14} coconut alcohol having a DP of 1 to 3 are preferred.
sugar surfactants of the type of the fatty acid N-alkylpolyhydroxyalkylamides, a nonionic surfactant of formula (T-III)

R⁶

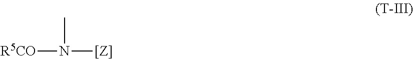

wherein R⁵CO is an aliphatic acyl residue having 6 to 22 carbon atoms, R⁶ is hydrogen, an alkyl or hydroxyalkyl residue having 1 to 4 carbon atoms, and [Z] is a linear or branched polyhydroxyalkyl residue having 3 to 12 carbon atoms and 3 to 10 hydroxyl groups. The fatty acid N-alkylpolyhydroxyalkylamides are known substances often obtained by reductive amination of a reducing sugar with ammonia, an alkylamine, or an alkanolamine, and subsequent acylation with a fatty acid, a fatty acid alkyl ester, or a fatty acid chloride. The fatty acid N-alkylpolyhydroxyalkylamides are preferably derived from reducing sugars having 5 or 6 carbon atoms, particularly from glucose. The preferred fatty acid N-alkylpolyhydroxyalkylamides therefore represent fatty acid N-alkylglucamides such as those reproduced by formula (T-IV):

R⁷CO—NR⁸—CH₂—(CHOH)₄—CH₂OH (T-IV).
It is preferable to use as fatty acid N-alkylpolyhydroxyalkylamides glucamides of formula (T-IV) wherein R⁸ is hydrogen or an alkyl group and R⁷CO is the acyl residue of hexanoic acid, octanoic acid, decanoic acid, lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid, or erucic acid, or industrial mixtures of those acids. Particularly preferred are fatty acid N-alkylglucamides of formula (T-IV) obtained by reductive amination of glucose with methylamine and subsequent acylation with lauric acid or C_{12/14} coconut fatty acid or a corresponding derivative. The polyhydroxyalkylamides can furthermore also be derived from maltose and palatinose.

Alkylene oxide addition products with saturated linear fatty alcohols and fatty acids having 2 to 100 mol ethylene oxide per mol fatty alcohol or fatty acid are very particularly preferred nonionic surfactants. Preparations having outstanding properties are likewise obtained if they contain, as nonionic surfactants, C₁₂ to C₃₀ fatty acid mono- and diesters of addition products of 1 to 30 mol ethylene oxide with glycerol and/or addition products of 5 to 60 mol ethylene oxide with castor oil and hardened castor oil.

For surfactants representing addition products of ethylene oxide and/or propylene oxide with fatty alcohols or derivatives of those addition products, both products having a “normal” homolog distribution and those having a restricted homolog distribution can be used. A “normal” homolog distribution is understood as mixtures of homologs obtained upon reaction of fatty alcohol and alkylene oxide using alkali metals, alkali metal hydroxides, or alkali metal alcoholates as catalysts. Restricted homolog distributions are obtained when, for example, hydrotalcites, alkaline-earth metal salts of ethercarboxylic acids, or alkaline-earth metal oxides, hydroxides, or alcoholates are used as catalysts. Use of products having a restricted homolog distribution can be preferred.

Very particularly preferably, agents according to the present invention contain as a surfactant at least one addition product of 15 to 100 mol ethylene oxide, particularly 15 to 50 mol ethylene oxide, with a linear or branched (particularly linear) fatty alcohol having 8 to 22 carbon atoms. This includes ceteareth-15, ceteareth-25, or ceteareth-50, marketed as Eumulgin® CS 15 (Cognis), Cremophor A25 (BASF SE), or Eumulgin® CS 50 (Cognis).

All anionic surface-active substances suitable for use on the human body are, in principle, appropriate as anionic surfactants. These have an anionic group imparting water solubility, for example a carboxylate, sulfate, sulfonate, or phosphate group, and a lipophilic alkyl group having approximately 8 to 30 carbon atoms. Glycol ether or polyglycol ether groups, ester, ether, and amide groups, and hydroxyl groups can additionally be contained in the molecule. Examples of suitable anionic surfactants are, each in the form of the sodium, potassium, and ammonium and mono-, di, and trialkanolammonium salts having 2 to 4 carbon atoms in the alkanol group:
linear and branched fatty acids having 8 to 30 carbon atoms (soaps);
ethercarboxylic acids of the formula R—O—(CH₂—CH₂O), —CH₂—COOH, wherein R is a linear alkyl group having 8 to 30 carbon atoms and x=0 or is 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
sulfosuccinic acid mono- and dialkyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups;
linear alkanesulfonates having 8 to 24 carbon atoms;
linear alpha-olefinsulfonates having 8 to 24 carbon atoms;
alpha-sulfo fatty acid methyl esters of fatty acids having 8 to 30 carbon atoms;
alkyl sulfates and alkyl polyglycol ether sulfates of the formula R—O—(CH₂—CH₂—O)ₓ—OSO₃H, wherein R is preferably a linear alkyl group having 8 to 30 carbon atoms and x=0 or is 1 to 12;
mixtures of surface-active hydroxysulfonates;
sulfated hydroxyalkylpolyethylene and/or hydroxyalkylenepropylene glycol ethers;
sulfonates of unsaturated fatty acids having 8 to 24 carbon atoms and 1 to 6 double bonds;
esters of tartaric acid and citric acid with alcohols, representing addition products of approximately 2 to 15 molecules of ethylene oxide and/or propylene oxide with fatty alcohols having 8 to 22 carbon atoms;
alkyl and/or alkenyl ether phosphates of formula (T-V)

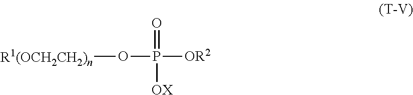

wherein R¹ preferably is an aliphatic hydrocarbon residue having 8 to 30 carbon atoms, R² is hydrogen, a (CH₂CH₂O)ₙR¹ residue, or X, n is numbers from 1 to 10, and X is hydrogen, an alkali or alkaline-earth metal, or NR³R⁴R⁵R⁶ where R³ to R⁶, mutually independently, are hydrogen or a C₁ to C₄ hydrocarbon residue;
sulfated fatty acid alkylene glycol esters of formula (T-VI)

R⁷CO(AlkKO)ₙSO₃M (T-VI)
wherein R⁷CO is a linear or branched, aliphatic, saturated and/or unsaturated acyl residue having 6 to 22 carbon atoms, Alk is CH₂CH₂, CHCH₃CH₂, and/or CH₂CHCH₃, n is a number from 0.5 to 5, and M is a cation;
monoglyceride sulfates and monoglyceride ether sulfates of formula (T-VII)

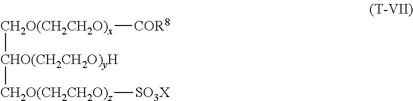

wherein R⁸CO is a linear or branched acyl residue having 6 to 22 carbon atoms, x, y, and z in total are 0 or a number from 1 to 30, preferably 2 to 10, and X is an alkali or alkaline-earth metal. Typical examples of monoglyceride (ether) sulfates suitable for purposes of the invention are the reaction products of lauric acid monoglyceride, coconut fatty acid monoglyceride, palmitic acid monoglyceride, stearic acid monoglyceride, oleic acid monoglyceride, and tallow fatty acid monoglyceride, as well as their ethylene oxide adducts with sulfur trioxide or chlorosulfonic acid in the form of their sodium salts. It is preferable to use monoglyceride sulfates of formula (T-VII) wherein R⁸CO is a linear acyl residue having 8 to 18 carbon atoms,
amide ethercarboxylic acids;
condensation products of C₈ to C₃₀ fatty alcohols with protein hydrolysates and/or amino acids and derivatives thereof, known to one skilled in the art as protein fatty acid condensates, such as Lamepon® grades, Gluadin® grades, Hostapon® KCG, or the Amisoft® grades.

Preferred anionic surfactants are alkyl sulfates, alkyl polyglycol ether sulfates, and ethercarboxylic acids having 10 to 18 carbon atoms in the alkyl group and up to 12 glycol ether groups in the molecule, sulfosuccinic acid mono- and dialkyl esters having 8 to 18 carbon atoms in the alkyl group, and sulfosuccinic acid monoalkylpolyoxyethyl esters having 8 to 18 carbon atoms in the alkyl group and 1 to 6 oxyethyl groups, monoglycerol disulfates, alkyl and alkenyl ether phosphates, and protein fatty acid condensates.

Cationic surfactants of the quaternary ammonium compound, esterquat, and amidoamine types are furthermore usable according to the present invention. Preferred quaternary ammonium compounds are ammonium halides, particularly chlorides and bromides such as alkyltrimethylammonium chlorides, dialkyldimethylammonium chlorides, and trialkylmethylammonium chlorides. The long alkyl chains of these surfactants preferably comprise 10 to 18 carbon atoms, as in cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. Further preferred cationic surfactants are the imidazolium compounds having the INCI designations Quaternium-27 and Quaternium-83.

“Zwitterionic surfactants” refers to those surface-active compounds having at least one quaternary ammonium group and at least one —COO⁽⁻⁾ or SO₃⁽⁻⁾ group. Particularly suitable zwitterionic surfactants are betaines such as N-alkyl-N,N-dimethylammonium glycinates, for example, cocalkyldimethylammonium glycinate, N-acylaminopropyl-N,N-dimethylammonium glycinates, for example, cocacylaminopropyldimethylammonium glycinate, and 2-alkyl-3-carboxymethyl-3-hydroxyethylimidazolines, each having 8 to 18 carbon atoms in the alkyl or acyl group, as well as cocacylaminoethylhydroxyethylcarboxymethyl glycinate. A preferred zwitterionic surfactant is the fatty acid amide derivative known by the INCI name Cocamidopropyl Betaine.

“Ampholytes” refer to those surface-active compounds having in the molecule, in addition to a C₈ to C₂₄ alkyl or acyl group, at least one free amino group and at least one —COOH or —SO₃H group and are capable of forming internal salts. Examples of suitable ampholytes are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, each having approximately 8 to 24 carbon atoms in the alkyl group. Particularly preferred ampholytes are N-cocalkylaminopropionate, cocacylaminoethylaminopropionate, and C₁₂₋₁₈ acyl sarcosine.

Agents according to the present invention can optionally additionally contain at least one film-forming polymer and/or setting polymer. These additional polymers are different from copolymer (a) and copolymer (b). The optionally added film-forming and/or setting polymers are preferably cationic and/or nonionic.

“Film-forming polymers” refer to those polymers that, upon drying, leave behind a continuous film on the skin, hair or nails. Film-formers of this kind can be used in a very wide variety of cosmetic products such as face masks, make-up, hair setting agents, hair sprays, hair gels, hair waxes, hair therapies, shampoos, or nail polishes. Particularly preferred are those polymers having sufficient solubility in alcohol or in water/alcohol mixtures so as to be present in completely dissolved form in the agent. The film-forming polymers can be of synthetic or natural origin.

“Film-forming polymers” further include those polymers that, when applied in a 0.01 to 20-wt % aqueous, alcoholic, or aqueous alcoholic solution, are capable of depositing a transparent polymer film on the hair.

Setting polymers contribute to hold and/or to building up hair volume and hair fullness of the overall hairstyle. These polymers are also film-forming polymers and are therefore generally typical substances for shape-imparting hair-treatment agents such as hair setting agents, hair foams, hair waxes, hair sprays. It is certainly possible for film formation to be localized, and for only a few fibers to be connected to one another.

The “curl retention” test is often used as a test method for the setting effect of a polymer.

In addition, the agent according to the present invention can contain at least one cationic film-forming and/or cationic setting polymer.

The additional cationic film-forming and/or cationic setting polymers comprise at least one structural unit having at least one permanently cationized nitrogen atom. “Permanently” cationized nitrogen atoms are to be understood as those nitrogen atoms having a positive charge and thereby form a quaternary ammonium compound. Quaternary ammonium compounds are usually produced by reaction of tertiary amines with alkylating agents such as methyl chloride, benzyl chloride, dimethyl sulfate, dodecyl bromide, as well as ethylene oxide. Depending on the tertiary amine used, the following groups are particularly known: alkylammonium compounds, alkenylammonium compounds, imidazolinium compounds, and pyridinium compounds.

Preferred agents for this embodiment contain the cationic film-forming and/or cationic setting polymers in an amount from 0.1 wt % to 20.0 wt %, preferably 0.2 wt % to 10.0 wt %, more preferably from 0.5 wt % to 5.0 wt %, based on total weight of the agent.

Cationic film-forming and/or cationic setting polymers can, according to the present invention, be chosen from cationic quaternized cellulose derivatives.

Those cationic quaternized celluloses having more than one permanent cationic charge in a side chain are generally advantageous for purposes of the embodiment.

To be emphasized among the cationic cellulose derivatives are those that are manufactured from a reaction of hydroxyethyl cellulose with a dimethyldiallylammonium reagent (in particular dimethyldiallylammonium chloride), if applicable in the presence of further reagents. Among these cationic celluloses, those cationic celluloses having the INCI name Polyquaternium-4 and marketed, for example, under the designations Celquat® H 100, Celquat® L 200 by the National Starch company, are particularly suitable.

Also suitable are those cationic film-forming and/or cationic setting polymers having at least one structural unit of formula (I) and at least one structural unit of formula (VI) and, if applicable, at least one structural unit of formula (V),

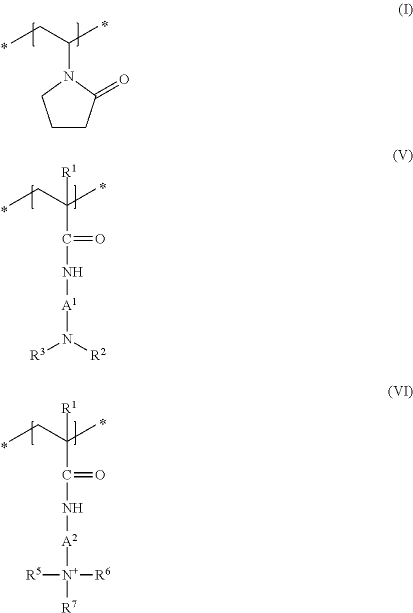

wherein
R¹ and R², mutually independently, are a hydrogen atom or a methyl group, A¹ and A², mutually independently, are an ethane-1,2-diyl, propane-1,3-diyl, or butane-1,4-diyl group,
R², R³, R⁵, and R⁶, mutually independently, are a (C₁ to C₄) alkyl group, and
R⁷ is a (C₈ to C₃₀) alkyl group.

All possible physiologically acceptable anions, for example, chloride, bromide, hydrogen sulfate, methyl sulfate, ethyl sulfate, tetrafluoroborate, phosphate, hydrogenphosphate, dihydrogenphosphate, or p-toluenesulfonate, triflate, serve to compensate for the positive charge of monomer (VI).

Suitable compounds include those obtainable commercially as
copolymers of dimethylaminoethyl methacrylate, quaternized with diethyl sulfate, with vinylpyrrolidone, having the INCI name Polyquaternium-11, under the designations Gafquat® 440, Gafquat® 734, Gafquat® 755 (each from the ISP company) and Luviquat PQ 11 PN (BASF SE),
copolymers of methacryloylaminopropyllauryldimethylammonium chloride with vinylpyrrolidone and dimethylaminopropyl methacrylamide, having the INCI name Polyquaternium-55, under the commercial names Styleze® W-10, Styleze® W-20 (ISP company).

Also serving as film-forming and/or setting polymers usable particularly preferably for purposes of the embodiment, selected from cationic polymers having at least one structural unit comprising a permanently cationized nitrogen atom are those cationic film-forming and/or cationic setting copolymers having at least one structural element of formula (M1)

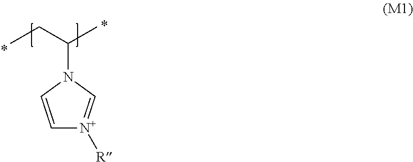

wherein R″ is a (C₁ to C₄) alkyl group, particularly a methyl group,
and additionally comprise at least one further cationic and/or nonionic structural element.

The statements made above apply with regard to compensation for the positive polymer charge.

It is preferred in turn according to the present invention in the context of this embodiment if the cationic film-forming and/or cationic setting polymer according to the present invention contains at least one copolymer (c1) that, alongside a structural element of formula (M1), additionally encompasses a structural element of formula (I)

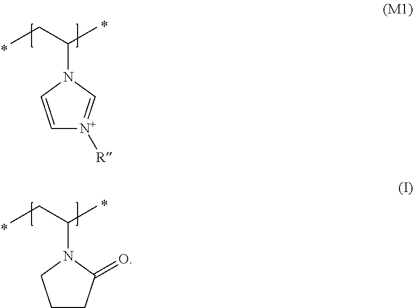

wherein R″ is a (C₁ to C₄) alkyl group, particularly a methyl group.

All possible physiologically acceptable anions, for example, chloride, bromide, hydrogen sulfate, methyl sulfate, ethyl sulfate, tetrafluoroborate, phosphate, hydrogenphosphate, dihydrogenphosphate, or p-toluenesulfonate, triflate, serve to compensate for the positive charge of monomer (c1).

Very particularly preferred cationic film-forming and/or cationic setting polymers contain as copolymers (c1) 10 to 30 mol %, preferably 15 to 25 mol %, and in particular 20 mol % structural units according to formula (M1), and 70 to 90 mol %, preferably 75 to 85 mol %, and in particular 80 mol % structural units according to formula (I).

It is particularly preferred in this context if copolymers (c1) contain, alongside polymer units resulting from incorporation of the aforesaid structural units according to formulas (M1) and (I) into the copolymer, a maximum of 5 wt %, preferably a maximum of 1 wt % of polymer units that are based on the incorporation of other monomers. Copolymers (c1) are preferably constructed exclusively from structural units of formula (M1) where R″=methyl and (I), and can be described by the general formula (Poly1)

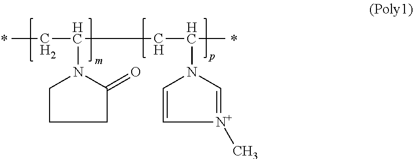

where m and p each vary depending on the molecular weight of the polymer and are not intended to signify that these are block copolymers. Structural units of formula (M1) and formula (I) can instead be present in statistically distributed fashion in the molecule.

If a chloride ion is used to compensate for the positive charge of the polymer of formula (Poly1), these N-methylvinylimidazole/vinylpyrrolidone copolymers are then referred to as Polyquaternium-16 according to INCI nomenclature, and are obtainable, for example, from BASF under the trade names Luviquat® Style, Luviquat® FC 370, Luviquat® FC 550, Luviquat® FC 905, and Luviquat® HM 552.

If a methosulfate is used to compensate for the positive charge of the polymer of formula (Poly1), these N-methylvinylimidazole/vinylpyrrolidone copolymers are then referred to as Polyquaternium-44 according to INCI nomenclature, and are obtainable, for example, from BASF under the trade names Luviquat® UltraCare.

Particularly preferred agents of this embodiment contain a copolymer (c1), particularly of formula (Poly 1), having molecular weights within a specific range. Agents in which copolymer (c1) has a molecular weight from 50 to 400 kDa, preferably from 100 to 300 kDa, more preferably from 150 to 250 kDa, and in particular from 190 to 210 kDa, are preferred here.

In addition to or instead of copolymer(s) (c1), agents according to the present invention can also contain copolymers (c2) that, proceeding from copolymer (c1), contain structural units of formula (II) as additional structural units:

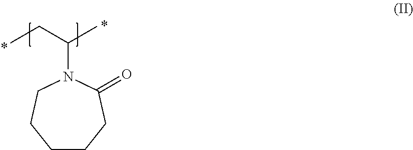

Further particularly preferred agents according to the present invention of this embodiment contain, as a cationic film-forming and/or cationic setting polymer, at least one copolymer (c2) having at least one structural unit according to formula (M1-a), at least one structural unit according to formula (I) and at least one according to formula (II)

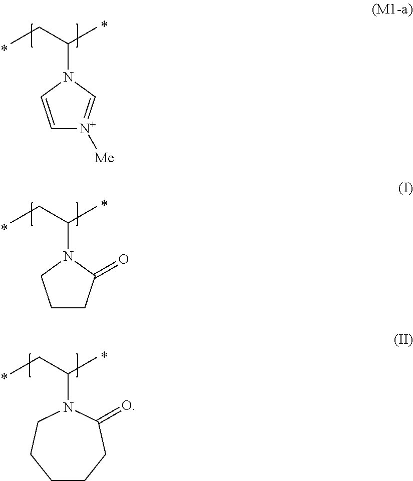

Here it is particularly preferred in this embodiment if copolymers (c2) contain, alongside polymer units resulting from incorporation of the aforesaid structural units according to formulae (M1-a), (I), and (II) into the copolymer, a maximum of 5 wt %, preferably a maximum of 1 wt % of polymer units based on the incorporation of other monomers. Copolymers (c2) are preferably constructed exclusively from structural units of formulas (M1-a), (I), and (II), and can be described by the general formula (Poly2)

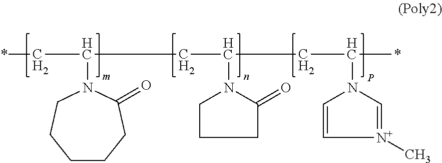

where m, n and p each vary depending on the molecular weight of the polymer and are not intended to signify that these are block copolymers. Structural units of the aforesaid formulae can instead be present in statistically distributed fashion in the molecule.

All possible physiologically acceptable anions, for example, chloride, bromide, hydrogen sulfate, methyl sulfate, ethyl sulfate, tetrafluoroborate, phosphate, hydrogenphosphate, dihydrogenphosphate, or p-toluenesulfonate, triflate, serve to compensate for the positive charge of monomer (c2).

If a methosulfate is used to compensate for the positive charge of the polymer of formula (Poly2), these N-methylvinylimidazole/vinylpyrrolidone/vinylcaprolactam copolymers are then referred to as Polyquaternium-46 according to INCI nomenclature, and are obtainable, for example, from BASF under the trade name Luviquat® Hold.

Very particularly preferred copolymers (c2) contain 1 to 20 mol %, preferably 5 to 15 mol %, and in particular 10 mol % structural units according to formula (M-1a), and 30 to 50 mol %, preferably 35 to 45 mol %, and in particular 40 mol % structural units according to formula (I), and 40 to 60 mol %, preferably 45 to 55 mol %, and in particular 60 mol % structural units according to formula (II).

Particularly preferred agents of this embodiment contain a copolymer (c2) having molecular weights within a specific range. Agents according to the present invention in which copolymer (c2) has a molecular weight from 100 to 1000 kDa, preferably from 250 to 900 kDa, more preferably from 500 to 850 kDa, and in particular from 650 to 710 kDa, are preferred.

In addition to or instead of copolymer(s) (c1) and/or (c2), the agents can also contain, as a cationic film-forming and/or cationic setting polymer, copolymers (c3) having as structural units structural units of formulas (M1-a) and (I), as well as further structural units from the group of the vinylimidazole units and further structural units from the group of the acrylamide and/or methacrylamide units.

Further particularly preferred agents of this embodiment contain, as an additional cationic film-forming and/or cationic setting polymer, at least one copolymer (c3) having at least one structural unit according to formula (M-1a), at least one further structural unit according to formula (I), at least one further structural unit according to formula (VII), and at least one further structural unit according to formula (VIII)

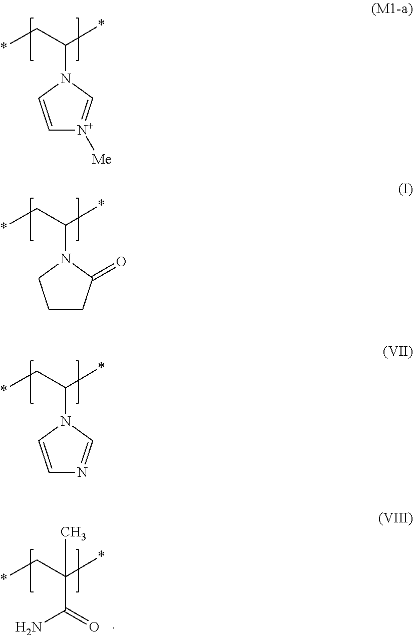

Here it is particularly preferred in the context of this embodiment if copolymers (c3) contain, in addition to polymer units resulting from incorporation of the aforesaid structural units according to formulas (M1-a), (I), (VII), and (VIII) into the copolymer, a maximum of 5 wt %, preferably a maximum of 1 wt %, of polymer units based on the incorporation of other monomers. Copolymers (c3) are preferably constructed exclusively from structural units of formulas (M 1-a), (I), (VII), and (VIII) and can be described by the general formula (Poly3)

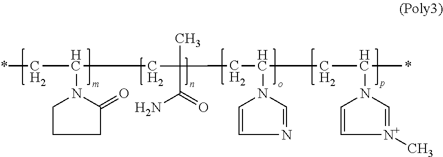

wherein m, n, o and p each vary depending on the molecular weight of the polymer and are not intended to signify that these are block copolymers. Structural units of formulas (M1-a), (I), (VII), and (VIII) can instead be present in statistically distributed fashion in the molecule.

All possible physiologically acceptable anions, for example, chloride, bromide, hydrogen sulfate, methyl sulfate, ethyl sulfate, tetrafluoroborate, phosphate, hydrogenphosphate, dihydrogenphosphate, or p-toluenesulfonate, triflate, serve to compensate for the positive charge of monomer (c3).

If a methosulfate is used to compensate for the positive charge of the polymer of formula (Poly3), these N-methylvinylimidazole/vinylpyrrolidone/vinylimidazole/methacrylamide copolymers are referred to as Polyquaternium-68 according to INCI nomenclature, and are obtainable, for example, from BASF under the trade name Luviquat® Supreme.

Very particularly preferred copolymers (c3) contain 1 to 12 mol %, preferably 3 to 9 mol %, and in particular 6 mol % structural units according to formula (M-1a), and 45 to 65 mol %, preferably 50 to 60 mol %, and in particular 55 mol % structural units according to formula (I), and 1 to 20 mol %, preferably 5 to 15 mol %, and in particular 10 mol % structural units according to formula (VII), and 20 to 40 mol %, preferably 25 to 35 mol %, and in particular 29 mol % structural units according to formula (VIII).

Particularly preferred agents of this embodiment contain a copolymer (c3) having molecular weights within a specific range. Agents according to the present invention in which copolymer (c3) has a molecular weight from 100 to 500 kDa, preferably from 150 to 400 kDa, more preferably from 250 to 350 kDa, and in particular from 290 to 310 kDa, are preferred here.

Among the additional cationic film-forming and/or setting polymers chosen from the cationic polymers having at least one structural element of the above formula (M1), those considered preferred are:

vinylpyrrolidone/1-vinyl-3-methyl-1H-imidazolium chloride copolymers (such as the one having the INCI name Polyquaternium-16 under the commercial designations Luviquat® Style, Luviquat® FC 370, Luviquat® FC 550, Luviquat® FC 905, and Luviquat® HM 552 (BASF SE)),
vinylpyrrolidone/1-vinyl-3-methyl-1H-imidazolium methyl sulfate copolymers (such as the one having the INCI name Polyquaternium-44 under the commercial designations Luviquat® Care (BASF SE)),
vinylpyrrolidone/vinylcaprolactam/1-vinyl-3-methyl-1H-imidazolium terpolymers (such as the one having the INCI name Polyquaternium-46 under the commercial designations Luviquat® Care or Luviquat® Hold (BASF SE)),
vinylpyrrolidone/methacrylamide/vinylimidazole/1-vinyl-3-methyl-1H-imidazolium methyl sulfate copolymers (such as the one having the INCI name Polyquaternium-68 under the commercial designations Luviquat® Supreme (BASF SE)), as well as mixtures of said polymers.

Agents according to the present invention can contain, as an additional film-forming and/or setting polymer, at least one nonionic film-forming and/or nonionic setting polymer. A “nonionic polymer” refers to a polymer that, in a protic solvent under standard conditions, carries substantially no structural units having permanently cationic or anionic groups that must be compensated for by counterions in order to obtain electroneutrality. “Cationic” groups include, for example, quaternized ammonium groups but not protonated amines. “Anionic” groups include, for example, carboxyl and sulfonic-acid groups.

The nonionic film-forming and/or nonionic setting polymers are present in the agent of this embodiment preferably in an amount from 0.1 wt % to 20.0 wt %, more preferably from 0.2 wt % to 15.0 wt %, very particularly preferably from 0.5 wt % to 10.0 wt %, based on total weight of the agent according to the present invention.

Those nonionic film-forming and/or nonionic setting polymers having at least one structural element of formula (M2)

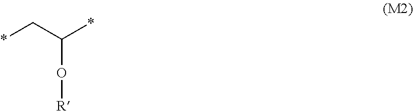

that carry, according to formula (M2), a hydrogen atom, an acetyl group, or a propanoyl group, particularly an acetyl group, as R′, are particularly suitable according to the present invention.

Nonionic film-forming and/or nonionic setting polymers are preferably chosen from at least one polymer of

homopolymers and nonionic copolymers of N-vinylpyrrolidone,

nonionic copolymers of isobutene.

Suitable polyvinylpyrrolidones include commercial products such as Luviskol® K 90 or Luviskol® K 85 of the BASF SE Company.

Suitable polyvinyl alcohols are marketed, for example, under the commercial designations Elvanol® by Du Pont, or Vinol® 523/540 by the Air Products Company.

Suitable polyvinyl acetate is marketed, for example, as an emulsion under the trade name Vinac® by the Air Products Company.

Agents having as a nonionic film-forming and/or nonionic setting polymer at least one polymer chosen from
polyvinylpyrrolidone,
copolymers of N-vinylpyrrolidone and vinyl esters of carboxylic acids having 2 to 18 carbon atoms, particularly of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di(C₁ to C₄) alkylamino-(C₂ to C₄) alkylacrylamide,
   are very particularly preferred according to the present invention.

Further preferred agents of this embodiment contain, as a nonionic film-forming and/or nonionic setting polymer, at least one copolymer (c4) having at least one further structural unit according to formula (I) and at least one structural unit according to formula (VII) and at least one structural unit according to formula (VIII)

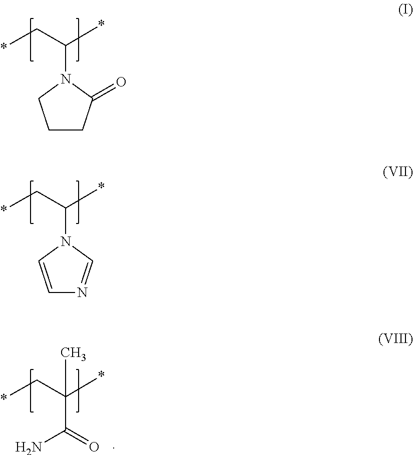

Here it is particularly preferred that these copolymers contain, in addition to polymer units resulting from incorporation of the aforesaid structural units according to formulas (I), (VII), and (VIII) into the copolymer, a maximum of 5 wt %, preferably a maximum of 1 wt % of polymer units based on incorporation of other monomers. Copolymers (c4) are preferably constructed exclusively from structural units of formulae (I), (VII), and (VIII) and can be described by the general formula (Poly4)

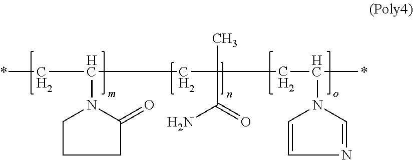

where m, n, o and p each vary depending on the molecular weight of the polymer and are not intended to signify that these are block copolymers. Structural units of formulae (I), (VII), and (VIII) can instead be present in statistically distributed fashion in the molecule.

A particularly preferred polymer is selected in this context from polymers having the INCI name VP/Methacrylamide/Vinyl Imidazole Copolymer, obtainable, for example, under the trade name Luviset Clear from the BASF SE Company.

Agents according to the present invention can contain organic solvents or a mixture of solvents having a boiling point under 400° C. as additional co-solvents in an amount from 0.1 to 15 wt %, preferably 1 to 10 wt %, based on total agent. Unbranched or branched hydrocarbons such as pentane, hexane, isopentane, and cyclic hydrocarbons such as cyclopentane and cyclohexane, are particularly suitable as additional co-solvents. Further particularly preferred water-soluble solvents are glycerol, ethylene glycol, butylene glycol, and propylene glycol, in an amount of up to 30 wt % based on total agent.

In particular, the addition of glycerol and/or propylene glycol and/or polyethylene glycol and/or polypropylene glycol increases the flexibility of the polymer film formed upon utilization of the agent according to the present invention. If a flexible hold is desired, the agents therefore contain preferably 0.01 to 30 wt % glycerol and/or propylene glycol and/or polyethylene glycol and/or polypropylene glycol, based on total agent.

The agents preferably have a pH from 2 to 11. Particularly preferably, the pH range is from 2 to 8. Reference to pH, for purposes of this document, refers to pH at 25° C. unless otherwise noted.

Agents according to the present invention can furthermore contain adjuvants and additives typically added to conventional styling agents.

Care-providing substances may be mentioned in particular as suitable adjuvants and additives.

The agent can contain as a care-providing substance, for example, at least one protein hydrolysate and/or a derivative thereof.

Protein hydrolysates are product mixtures obtained by acid-, base-, or enzyme-catalyzed breakdown of proteins. The term “protein hydrolysates” according to the present invention refers to total hydrolysates as well as individual amino acids and derivatives thereof, as well as mixtures of different amino acids. Polymers constructed from amino acids and amino-acid derivatives are also understood according to the present invention under the term “protein hydrolysates”. Included among the latter are, for example, polyalanine, polyasparagine, polyserine, etc. Further examples of compounds usable according to the present invention are L-alanyl-L-proline, polyglycine, glycyl-L-glutamine, or D/L-methionine-S-methylsulfonium chloride. β-Amino acids and their derivatives, such as β-alanine, anthranilic acid, or hippuric acid, can of course also be used according to the present invention. The molecular weight of protein hydrolysates usable according to the present invention is from 75 (the molecular weight of glycine) to 200,000; the molecular weight is preferably 75 to 50,000 Dalton, and very particularly preferably 75 to 20,000 Dalton.

According to the present invention, protein hydrolysates of both vegetable and animal origin, or of marine or synthetic origin, can be used.

Animal protein hydrolysates include hydrolysates of elastin, collagen, keratin, silk, and milk protein, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Use of silk protein hydrolysates is of particular interest. “Silk” is understood as the fibers of the cocoon of the mulberry silkworm (Bombyx mori L.). The raw silk fiber is made up of a double thread of fibroin. Sericin serves as a glue substance holding this double thread together. Silk is made up of 70 to 80 wt % fibroin, 19 to 28 wt % sericin, 0.5 to 1 wt % fat, and 0.5 to 1 wt % coloring agents and mineral constituents.

The essential constituents of sericin are approximately 46 wt % hydroxyamino acids. Sericin is made up of a group of 5 to 6 proteins. The essential amino acids of sericin are serine (Ser, 37 wt %), aspartate (Asp, 26 wt %), glycine (Gly, 17 wt %), alanine (Ala), leucine (Leu), and tyrosine (Tyr).

Water-insoluble fibroin is included among the scleroproteins having a long-chain molecular structure. The principal constituents of fibroin are glycine (44 wt %), alanine (26 wt %), and tyrosine (13 wt %). A further essential structural feature of fibroin is the hexapeptide sequence Ser-Gly-Ala-Gly-Ala-Gly.

It is technically simple to separate the two silk proteins from one another. It is therefore not surprising that both sericin and fibroin are known, each individually, as raw materials for use in cosmetic products. Protein hydrolysates and protein derivatives based on the respective individual silk proteins are also known raw materials in cosmetic agents. For example, sericin as such is marketed by Pentapharm Ltd. as a commercial product with the designation Sericin Code 303-02. Fibroin is offered far more frequently on the market as a protein hydrolysate, at various molecular weights. These hydrolysates are marketed in particular as “silk hydrolysates.”Hydrolyzed fibroin having average molecular weights from 350 to 1000 is marketed, for example, under the commercial designation Promois® Silk.

The positive properties of the silk protein derivatives from sericin and fibroin, individually for each one, are known in the literature. For example, the sales brochure of the Pentapharm Company describes the cosmetic effects of sericin on the skin as irritation-soothing, hydrating, and film-forming. The effect of a fibroin derivative is described, for example in DE 31 39 438 A1, as providing care to and revival of the hair. According to DE 102 40 757 A1, with the simultaneous use of sericin and fibroin, or derivatives and/or hydrolysates thereof, it is furthermore possible to achieve a synergistic enhancement of the positive effects of the silk proteins and their derivatives.

It is therefore preferred to use in the agent as a silk protein hydrolysate, an active-substance complex (A) made up of the active substance (A1) chosen from sericin, sericin hydrolysates, and/or derivatives thereof, as well as mixtures thereof, and an active substance (A2) chosen from fibroin and/or fibroin hydrolysates and/or derivatives thereof and/or mixtures thereof.

The active-substance complex (A) significantly improves, in synergistic fashion, the essential internal and external structural features presented above, as well as the strength and elasticity of human hairs.

Particularly good care-providing properties can be achieved if one of the two active-substance components of the active-substance complex (A) is used in the natural or, if need be, solubilized form. It is also possible to utilize a mixture of several active substances (A1) and/or (A2).

It can be preferred for the two active substances (A1) and (A2) to be used in the agents according to the present invention at a ratio from 10:90 to 70:30, particularly 15:85 to 50:50, and very particularly 20:80 to 40:60, based on their respective active-substance contents.

Protein hydrolysates of vegetable origin (e.g., soy, almond, bean, potato, and wheat protein hydrolysates) are obtainable, for example, under the trademarks Gluadin® (Cognis), DiaMin® (Diamalt), Lexein® (Inolex), Hydrosoy® (Croda), Hydrolupin (Croda), Hydrosesame® (Croda), Hydrotritium® (Croda), and Crotein® (Croda).

Although use of protein hydrolysates as such is preferred, it is also optionally possible to use instead of them, if applicable, amino-acid mixtures obtained in different fashion. It is likewise possible to use derivatives of protein hydrolysates, for example, in the form of their fatty acid condensation products. Such products are marketed, for example, under the designations Lamepon® (Cognis), Lexein® (Inolex), Crolastin® (Croda), Crosilk® (Croda), or Crotein® (Croda).

The teaching according to the present invention of course encompasses all isomeric forms, such as cis-trans isomers, diastereomers, and chiral isomers. It is also possible according to the present invention to use a mixture of several protein hydrolysates.

Protein hydrolysates can be present in the agents, for example, in concentrations from 0.01 wt % to 20 wt %, preferably 0.05 wt % to 15 wt %, and very particularly preferably from 0.05 wt % to 5 wt %, based on total application preparation.

The agent according to the present invention can further contain at least one vitamin, provitamin, vitamin precursor, and/or derivatives thereof as a care-providing substance.

Those vitamins, provitamins, and vitamin precursors that are usually assigned to groups A, B, C, E, F, and H are preferred according to the present invention.

The group of substances referred to as vitamin A includes retinol (vitamin A₁) as well as 3,4-didehydroretinol (vitamin A₂). β-Carotene is the provitamin of retinol. Vitamin A components that are suitable according to the present invention are, for example, vitamin A acid and its esters, vitamin A aldehyde, and vitamin A alcohol, as well as its esters thereof such as the palmitate and acetate. The agents contain the vitamin A component preferably in quantities from 0.05 to 1 wt % based on the entire application preparation.

Members of the vitamin B group or vitamin B complex include, among others:
Vitamin B₁ (thiamine)
Vitamin B₂ (riboflavin)
Vitamin B₃. The compounds nicotinic acid and nicotinic acid amide (niacinamide) are often listed under this designation. Nicotinic acid amide is preferred according to the present invention; it is contained in the agents according to the present invention preferably in quantities from 0.05 to 1 wt % based on total application preparation.
Vitamin B₅ (pantothenic acid, panthenol, and pantolactone). Panthenol and/or pantolactone are preferably used in the context of this group. Derivatives of panthenol usable according to the present invention are, in particular, the esters and ethers of panthenol as well as cationically derivatized panthenols. Individual representatives are, for example, panthenol triacetate, panthenol monoethyl ether and the monoacetate thereof, and cationic panthenol derivatives. The aforesaid compounds of the vitamin B₅ type are contained in the agents according to the present invention preferably in quantities from 0.05 to 10 wt % based on total application preparation. Quantities from 0.1 to 5 wt % are particularly preferred.
Vitamin B₆ (pyridoxine as well as pyridoxamine and pyridoxal). The aforesaid compounds of the vitamin B₆ type are contained in the agents according to the present invention preferably in quantities from 0.01 to 5 wt % based on the entire application preparation. Quantities from 0.05 to 1 wt % are particularly preferred.

Vitamin C (ascorbic acid). Vitamin C is utilized in the agents according to the present invention preferably in quantities from 0.1 to 3 wt % based on the entire application preparation. Utilization in the form of the palmitic acid ester, the glucosides or phosphates can be preferred. Utilization in combination with tocopherols can likewise be preferred.

Vitamin E (tocopherols, in particular α-tocopherol). Tocopherol and its derivatives, which include in particular the esters such as the acetate, nicotinate, phosphate, and succinate, are contained in the agents according to the present invention preferably in quantities from 0.05 to 1 wt % based on the entire application preparation.

Vitamin F. The term “vitamin F” is usually understood as essential fatty acids, in particular linoleic acid, linolenic acid, and arachidonic acid.

Vitamin H. This refers to (3aS,4S,6aR)-2-oxohexahydrothienol[3,4-d]-imidazole-4-valeric acid, for which the trivial name “biotin” has nevertheless since become established. Biotin is contained in the agents according to the present invention preferably in quantities from 0.0001 to 1.0 wt %, particularly from 0.001 to 0.01 wt %, based on total application preparation.

Agents according to the present invention preferably contain vitamins, provitamins, and vitamin precursors from groups A, B, C, E and H.

Panthenol, pantolactone, pyridoxine and its derivatives, as well as nicotinic acid amide and biotin, are particularly preferred.

D-panthenol is used very particularly preferably as a care-providing substance, if applicable in combination with at least one of the aforesaid silicone derivatives.

Like the addition of glycerol and/or propylene glycol, the addition of panthenol also increases the flexibility of the polymer film formed upon utilization of the agent according to the present invention. If a particularly flexible hold is desired, agents according to the present invention can thus contain panthenol instead of or in addition to glycerol and/or propylene glycol. In a preferred embodiment the agents contain panthenol, preferably in an amount from 0.05 to 10 wt %, more preferably 0.1 to 5 wt %, based on total agent.

Agents according to the present invention can further contain at least one plant extract as a care-providing substance.

These extracts are usually produced by extraction of the entire plant. In individual cases, however, it may also be preferred to produce the extracts exclusively from blossoms and/or leaves of the plant.

Regarding plant extracts usable according to the present invention, reference is made to extracts listed in the table beginning on page 44 of the 3rd edition of the “Guideline for declaring the contents of cosmetic agents” [Leitfaden zur Inhaltsstoffdeklaration kosmetischer Mittel] published by the Association of the personal hygiene and washing agents industry [Industrieverband Körperpflege- and Waschmittel e.V. (IKW)], Frankfurt.

According to the present invention, extracts from green tea, oak bark, nettle, hamamelis, hops, henna, chamomile, burdock root, horsetail, hawthorn, linden blossoms, almond, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, meristem, ginseng, and ginger root are especially preferred.

Particularly preferred are the extracts from green tea, oak bark, nettle, hamamelis, hops, chamomile, burdock root, horsetail, linden blossoms, almond, aloe vera, coconut, mango, apricot, lemon, wheat, kiwi fruit, melon, orange, grapefruit, salvia, rosemary, birch, lady's-smock, wild thyme, yarrow, restharrow, meristem, ginseng, and ginger root.

The extracts from green tea, almond, aloe vera, coconut, mango, apricot, lemon, wheat, kiwi fruit, and melon are very particularly suitable.

Water, alcohols, and mixtures thereof can be used as extraction agents for manufacturing the aforesaid plant extracts. Among the alcohols, lower alcohols such as ethanol and isopropanol, particularly polyvalent alcohols such as ethylene glycol and propylene glycol, both as the only extraction agent and mixed with water, are preferred. Plant extracts based on water/propylene glycol at a ratio from 1:10 to 10:1 have proven particularly suitable. It is possible according to the present invention to use hydrous plant extracts in the context of the predefined water quantity. This is not, however, preferred according to the present invention.

According to the present invention, plant extracts can be used in both pure and diluted form. If they are used in diluted form, they usually contain approximately 2 to 80 wt % active substance, and contain as a solvent the extraction agent or extraction agent mixture used to obtain them.

It may furthermore be preferred to use mixtures of several, particularly two, different plant extracts in the agents.

Agents according to the present invention contain these care-providing substances preferably in amounts from 0.001 to 2 wt %, particularly 0.01 to 0.5 wt %, based on total application preparation.

Mono- or oligosaccharides can also be used as a care-providing substance in agents according to the present invention.

Both monosaccharides and oligosaccharides, for example, raw sugar, milk sugar, and raffinose, can be used. Use of monosaccharides is preferred. Among the monosaccharides, those compounds containing 5 or 6 carbon atoms are preferred.

Suitable pentoses and hexoses include ribose, arabinose, xylose, lyxose, allose, altrose, glucose, mannose, gulose, idose, galactose, talose, fucose and fructose. Arabinose, glucose, galactose and fructose are carbohydrates that are preferably used; it is very particularly preferred to use glucose, which is suitable both in the D-(+) or L-(−) configuration or as a racemate.

Derivatives of these pentoses and hexoses, such as the corresponding -onic and -uronic acids (sugar acids), sugar alcohols, and glycosides, can also be used according to the present invention. Preferred sugar acids are gluconic acid, glucuronic acid, saccharic acid, mannosaccharic acid, and mucic acid. Preferred sugar alcohols are sorbitol, mannitol, and dulcitol. Preferred glycosides are the methylglucosides.

Because the mono- or oligosaccharides that are used are usually obtained from natural raw materials such as starch, they typically exhibit the configurations corresponding to those raw materials (e.g., D-glucose, D-fructose and D-galactose).

The mono- or oligosaccharides are present in agents according to the present invention preferably in an amount from 0.1 to 8 wt %, more preferably from 1 to 5 wt %, based on total application preparation.

Although each of the aforesaid care-providing substances already yields a satisfactory result of itself, all embodiments in which the agent contains multiple care-providing substances, including from different groups, are also encompassed within the scope of the present invention.

The addition of a UV filter allows both the agents themselves and the treated fibers to be protected from damaging influences of UV radiation. At least one UV filter is therefore preferably added to the agent. Suitable UV filters are not subject to any general restrictions in terms of their structure and their physical properties. Instead, all UV filters usable in the cosmetics sector, whose absorption maximum lies in the UVA (315 to 400 nm) UVB (280 to 315 nm), or UVC (<280 nm) regions, are suitable. UV filters having an absorption maximum in the UVB region, particularly in the region from approximately 280 to approximately 300 nm, are particularly preferred.

Preferred UV filters can be chosen from substituted benzophenones, p-aminobenzoic acid esters, diphenylacrylic acid esters, cinnamic acid esters, salicylic acid esters, benzimidazoles, and o-aminobenzoic acid esters.

UV filters usable according to the present invention include 4-aminobenzoic acid, N,N,N-trimethyl-4-(2-oxoborn-3-ylidenemethyl)aniline methylsulfate, 3,3,5-trimethylcyclohexyl salicylate (Homosalate), 2-hydroxy-4-methoxybenzophenone (Benzophenone-3; Uvinul® M 40, Uvasorb® MET, Neo Heliopan® BB, Eusolex® 4360), 2-phenylbenzimidazole-5-sulfonic acid and potassium, sodium, and triethanolamine salts thereof (phenylbenzimidazolesulfonic acid; Parsol® HS; Neo Heliopan® Hydro), 3,3′-(1,4-phenylenedimethylene)-bis(7,7-dimethyl-2-oxo-bicyclo-[2.2.1]hept-1-yl-methanesulfonic acid) and salts thereof, 1-(4-tert-butylphenyl)-3-(4-methoxyphenyl)propane-1,3-dione (butylmethoxydibenzoylmethane; Parsol® 1789, Eusolex® 9020), α-(2-oxoborn-3-ylidene)toluene-4-sulfonic acid and salts thereof, ethoxylated 4-aminobenzoic acid ethyl ester (PEG-25 PABA; Uvinul® P 25), 4-dimethylaminobenzoic acid 2-ethylhexyl ester (Octyl Dimethyl PABA; Uvasorb® DMO, Escalol® 507, Eusolex® 6007), salicylic acid 2-ethylhexyl ester (Octyl Salicylate; Escalol® 587, Neo Heliopan® OS, Uvinul® 018), 4-methoxycinnamic acid isopentyl ester (Isoamyl p-Methoxycinnamate; Neo Heliopan® E 1000), 4-methoxycinnamic acid 2-ethylhexyl ester (Octyl Methoxycinnamate; Parsol® MCX, Escalol® 557, Neo Heliopan® AV), 2-hydroxy-4-methoxybenzophenone-5-sulfonic acid and the sodium salt thereof (Benzophenone-4; Uvinul® MS 40; Uvasorb® S 5), 3-(4′-methylbenzylidene) D,L-camphor (4-Methylbenzylidene Camphor; Parsol® 5000, Eusolex® 6300), 3-benzylidene camphor (3-Benzylidene Camphor), 4-isopropylbenzylsalicylate, 2,4,6-trianilino-(p-carbo-2′-ethylhexyl-1′-oxi)-1,3,5-triazine, 3-imidazol-4-ylacrylic acid and ethyl esters thereof, polymers of N-{(2 and 4)-[2-oxoborn-3-ylidenemethyl]benzyl}acrylamide, 2,4-dihydroxybenzophenone (Benzophenone-1; Uvasorb® 20 H, Uvinul® 400), 1,1′-diphenylacrylonitrilic acid 2-ethylhexyl ester (Octocrylene; Eusolex® OCR, Neo Heliopan® Type 303, Uvinul® N 539 SG), o-aminobenzoic acid menthyl ester (Menthyl Anthranilate; Neo Heliopan® MA), 2,2′,4,4′-tetrahydroxybenzophenone (Benzophenone-2; Uvinul® D-50), 2,2′-dihydroxy-4,4′-dimethoxybenzophenone (Benzophenone-6), 2,2′-dihydroxy-4,4′-dimethoxybenzophenone-5-sodiumsulfonate, and 2-cyano-3,3-diphenylacrylic acid 2′-ethylhexyl ester. The use of 4-methoxycinnamic acid isopentyl ester (Isoamyl p-Methoxycinnamate; Neo-Heliopan® E 1000) is particularly preferred.

UV filters whose molar extinction coefficient at the absorption maximum is above 15,000, particularly above 20,000, are preferred.

It has furthermore been found that with structurally similar UV filters, in the context of the teaching of the present invention the water-insoluble compound in many cases exhibits the greater effectiveness as compared with those water-soluble compounds that differ from it by having one or more additionally ionic groups. In the context of the invention, those UV filters of which no more than 1 wt %, particularly no more than 0.1 wt %, dissolves in water at 20° C., are understood to be “water-insoluble.” These compounds should furthermore be soluble at a proportion of at least 0.1, particularly at least 1 wt %, in common cosmetic oil components at room temperature. Use of water-insoluble UV filters can therefore be preferred.

The UV filters are present usually in quantities from 0.01 to 5 wt %, based on total application preparation. Quantities from 0.1 to 2.5 wt % are preferred.

In a particular embodiment, the agent further contains one or more substantive dyes. This allows keratinic fibers treated with the agent to be not only temporarily structured, but also dyed at the same time. This can be particularly desirable when what is desired is only temporary coloring (for example, with conspicuous “fashion” colors) which can be removed from the keratinic fibers simply by washing.

Substantive dyes are usually nitrophenylenediamines, nitroaminophenols, azo dyes, anthraquinones, or indophenols. Preferred substantive dyes are compounds known under the international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 11, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52, as well as 1,4-diamino-2-nitrobenzene, 2-amino-4-nitrophenol, 1,4-bis-(β-hydroxyethyl)amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-1-hydroxy-4-nitrobenzene.

It is preferred to use cationic substantive dyes. Particularly preferred in this context are
(a) cationic triphenylmethane dyes such as Basic Blue 7, Basic Blue 26, Basic Violet 2, and Basic Violet 14;
(b) aromatic systems substituted with a quaternary nitrogen group, such as Basic Yellow 57, Basic Red 76, Basic Blue 99, Basic Brown 16, and Basic Brown 17; and
(c) substantive dyes containing a heterocycle having at least one quaternary nitrogen atom, as recited for example in claims 6 to 11 in EP-A2-998 908.

Very particularly preferred cationic substantive dyes of group (c) are dyes known by the designations Basic Yellow 87, Basic Orange 31, and Basic Red 51.

Cationic substantive dyes marketed under the trademark Arianor® are, according to the present invention, likewise very particularly preferred cationic substantive dyes.

Agents in accordance with this embodiment contain substantive dyes preferably in a quantity from 0.001 to 20 wt %, based on total agent.

In addition, agents according to the present invention can also contain dyes occurring in nature, such as those contained in henna red, henna neutral, henna black, chamomile blossom, sandalwood, black tea, buckthorn bark, salvia, logwood, madder root, catechu, Spanish cedar, and alkanna root.

It is not necessary for the substantive dyes to represent homogeneous compounds in each case. The agents can instead, depending on the manufacturing methods for the individual dyes, also contain further components in subordinate quantities, provided they do not disadvantageously influence the styling result or do not have to be excluded for other (e.g., toxicological) reasons.

Preferably, the agents are free of oxidizing dye precursor products. Oxidizing dye precursor products are divided into developer components and coupler components. The developer components form the actual dyes with one another under the influence of oxidizing agents or atmospheric oxygen, or by coupling with one or more coupler components.

Alongside the aforesaid components, the agents can also contain all active substances, additives, and adjuvants known for corresponding cosmetic agents.

Further active substances, adjuvants, and additives include:
thickening agents such as agar-agar, guar gum, alginates, xanthan gum, gum arabic, karaya gum, locust bean flour, linseed gums, dextrans, cellulose derivatives (e.g., methyl cellulose, hydroxyalkyl cellulose, and carboxymethyl cellulose), starch fractions and derivatives such as amylose, amylopectin, and dextrins, clays such as bentonite, entirely synthetic hydrocolloids such as poly(vinylalcohol), and optionally crosslinked polyacrylates;
structuring agents such as maleic acid and lactic acid;
perfume oils, dimethylisosorbide, and cyclodextrins;
defoamers such as silicones;
dyes for coloring the agent;
anti-dandruff ingredients such as piroctone olamine, zinc omadine, and climbazole;
substances for adjusting pH, such as usual acids, particularly edible acids, and bases;
consistency agents such as sugar esters, polyol esters, or polyolalkyl ethers;
complexing agents such as EDTA, NTA, β-alaninediacetic acid, and phosphonic acids;
swelling and penetrating substances such as glycerol, propylene glycol monoethyl ether, carbonates, hydrogencarbonates, guanidines, ureas, and primary, secondary, and tertiary phosphates;
opacifiers such as latex, styrene/PVP copolymers, and styrene/acrylamide copolymers;
luster agents such as ethylene glycol mono- and distearate, and PEG-3 distearate;
preservatives;
antioxidants.

With regard to further optional components as well as the quantities of those components that are used, reference is made expressly to relevant manuals known to one skilled in the art.

Agents according to the present invention can be formulated in any form usual for styling agents, for example, in the form of solutions that can be applied onto the hair as a lotion or a pump or aerosol spray, or other preparations suitable for application to the hair.

Agents according to the present invention are preferably configured as a pump spray, aerosol spray, pump foam, or aerosol foam.

For this purpose, the agents are packaged in a delivery apparatus represented by either a pressurized gas container additionally filled with a propellant (“aerosol container”), or a non-aerosol container.

The pressurized gas container with which a product is distributed through a valve as a result of the internal gas pressure of the container is referred to as an “aerosol container”. A “non-aerosol container” is defined, conversely to the “aerosol” definition, as a vessel under standard pressure with which a product is distributed by mechanical action by a pump system.

Agents according to the present invention are present preferably as an aerosol hair foam or aerosol hair spray. The agent therefore preferably additionally contains at least one propellant.

Agents that are present in the form of an aerosol product can be manufactured in usual fashion. Typically, all ingredients of the agent except for the propellant are introduced into a suitable pressure-resistant container. The latter is then sealed with a valve. Lastly, the desired quantity of propellant is introduced using conventional techniques.

When in the form of an aerosol spray, propellants suitable according to the present invention are chosen, for example, from N₂O, dimethyl ether, CO₂, air, alkanes having 3 to 5 carbon atoms such as propane, n-butane, isobutane, n-pentane, and isopentane, and mixtures thereof. Dimethyl ether, propane, n-butane, isobutane, and mixtures thereof are preferred. According to a preferred embodiment, the aforesaid alkanes, mixtures of the aforesaid alkanes, or mixtures of the aforesaid alkanes with dimethyl ether are used as the only propellant. The invention also includes the concurrent use of propellants of the chlorofluorocarbon type, particularly fluorocarbons.

The propellant is present in agents of the aerosol spray embodiment preferably in an amount from 30 to 60 wt %, based on total weight of agent.

Very preferably, mixtures of propane and butane are used as the only propellant, at a weight ratio of propane to butane from 20 to 80 to 15 to 85. These mixtures are used in the agents preferably in a quantity from 30 to 55 wt %, based on total weight of the agent. “Butane” is understood according to the present invention as n-butane, isobutane, and mixtures of n-butane and isobutane.

A particularly preferred aerosol spray of this invention is an agent, packaged in an aerosol container, containing in a cosmetically acceptable carrier:
i) 1.0 wt % to 2.8 wt %, preferably from 1.5 wt % to 2.8 wt %, based on total weight of the cosmetic agent, of at least one copolymer (a) having at least one structural unit of formula (I), at least one structural unit of formula (II) and at least one structural unit of formula (III-1)

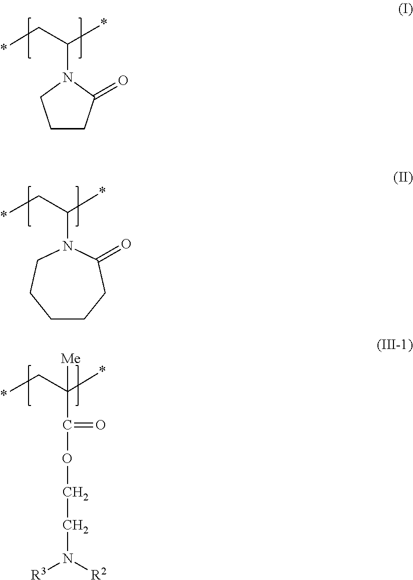

wherein R² and R³, mutually independently, are a (C₁ to C₄) alkyl group, and
ii) 0.4 wt % to 1.4 wt %, preferably from 0.6 wt % to 1.3 wt %, based on total weight of the cosmetic agent, of at least one copolymer (b) having at least one structural unit of formula (I), at least one structural unit of formula (II) and at least one structural unit of formula (IV-1)

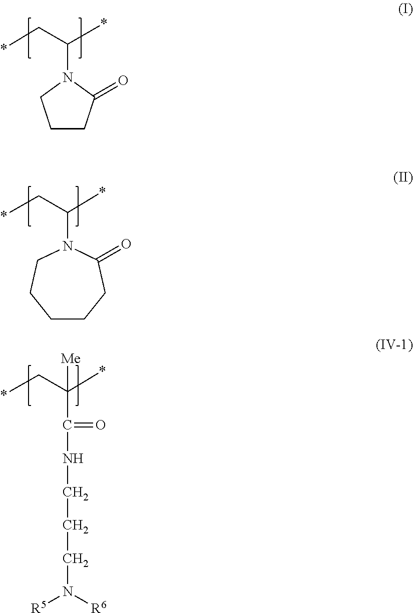

wherein R² and R³, mutually independently, are a (C₁ to C₄) alkyl group, and
iii) at least one propellant (particularly as the only propellant, 30 to 55 wt % of a mixture of propane and butane at a weight ratio of propane to butane from 20 to 80 to 15 to 85),
   wherein copolymer (a) and copolymer (b) are present in a weight ratio range (i) to (ii) from 1 to 1 to 1 to 5, particularly from 1 to 1.5 to 1 to 2.5.

Examples of (C₁ to C₄) alkyl groups according to formulae (III-1) and (IV-1) are methyl, ethyl, isopropyl, n-propyl, n-butyl, sec-butyl, isobutyl, and tert-butyl.

A very particularly preferred aerosol spray is an agent packaged in an aerosol container containing in a cosmetically acceptable carrier:
i) 1.0 wt % to 2.8 wt %, preferably from 1.5 wt % to 2.8 wt %, based on total weight of the cosmetic agent, of at least one terpolymer of N-vinylpyrrolidone, N-vinylcaprolactam, and N,N-dimethylaminoethyl methacrylate as copolymer (a), and
ii) 0.4 wt % to 1.4 wt %, preferably from 0.6 wt % to 1.3 wt %, based on total weight of the cosmetic agent, of at least one terpolymer of N-vinylpyrrolidone, N-vinylcaprolactam, and N,N-dimethylaminopropyl methacrylamide as copolymer (b), and
iii) at least one propellant (particularly as the only propellant, 30 to 55 wt % of a mixture of propane and butane at a weight ratio of propane to butane from 20 to 80 to 15 to 85),
   wherein copolymer (a) and copolymer (b) are present in a weight ratio range (i) to (ii) from 1 to 1 to 1 to 5, particularly from 1 to 1.5 to 1 to 2.5.

For a given spray apparatus, the size of the aerosol droplets and the respective size distribution can be adjusted by the quantitative ratio between the propellant and the other constituents of the preparations.

The spray rate of sprays according to the present invention is preferably 6.5 to 10.0 g/10 s.

Particularly preferred agents according to the present invention (aerosol sprays) are packaged in an aerosol container having a stem valve with a stem orifice having a diameter from 0.27 to 0.35 mm. Such valves are marketed, for example, by the Coster company as model KE or model KEN valves.

In addition, nozzles configured as swirl nozzles are particularly suitable as an aerosol spray for the present invention. Swirl nozzles comprise a conduit guide, known to one skilled in the art, to the center inside the nozzle. Swirl nozzles of this kind are marketed, for example, under the designation “standard micromist insert” by the Coster company.

A particularly preferred aerosol spray of this invention is an agent packaged in an aerosol container containing in a cosmetically acceptable carrier:
i) 1.0 wt % to 2.8 wt %, preferably from 1.5 wt % to 2.8 wt %, based on total weight of the cosmetic agent, of at least one copolymer (a) having at least one structural unit of formula (I), at least one structural unit of formula (II) and at least one structural unit of formula (III-1)

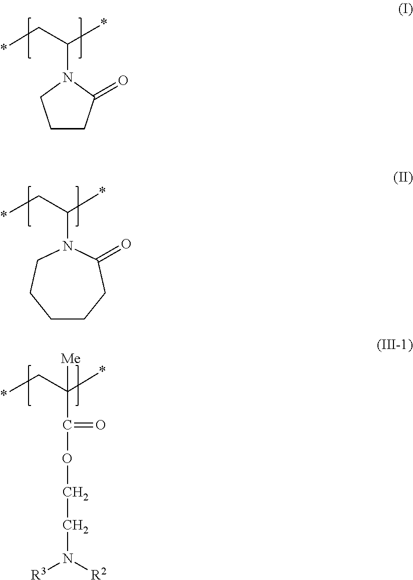

wherein R² and R³, mutually independently, are a (C₁ to C₄) alkyl group, and
ii) 0.4 wt % to 1.4 wt %, preferably from 0.6 wt % to 1.3 wt %, based on total weight of the cosmetic agent, of at least one copolymer (b) having at least one structural unit of formula (I), at least one structural unit of formula (II) and at least one structural unit of formula (IV-1)

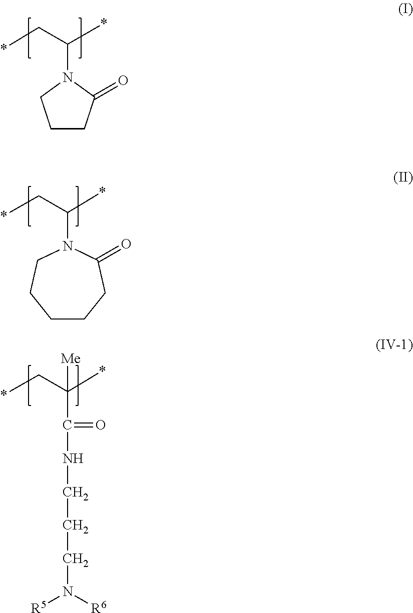

wherein R⁵ and R⁶, mutually independently, denote a (C₁ to C₄) alkyl group, and
iii) at least one propellant (particularly as the only propellant, 30 to 55 wt % of a mixture of propane and butane at a weight ratio of propane to butane from 20 to 80 to 15 to 85),
   the aerosol container comprising a stem valve having a stem orifice with a diameter from 0.27 to 0.35 mm, and preferably additionally bearing a swirl nozzle.

A very particularly preferred aerosol spray of this invention is therefore an agent, packaged in an aerosol container containing in a cosmetically acceptable carrier:
i) 10. wt % to 2.8 wt %, preferably from 1.5 wt % to 2.8 wt %, based on total weight of the cosmetic agent, of at least one terpolymer of N-vinylpyrrolidone, N-vinylcaprolactam, and N,N-dimethylaminoethyl methacrylate as copolymer (a), and
ii) 0.4 wt % to 1.4 wt %, preferably from 0.6 wt % to 1.3 wt %, based on total weight of the cosmetic agent, of at least one terpolymer of N-vinylpyrrolidone, N-vinylcaprolactam, and N,N-dimethylaminopropyl methacrylamide as copolymer (b), and
iii) at least one propellant (particularly, as the only propellant, 30 to 55 wt % of a mixture of propane and butane at a weight ratio of propane to butane from 20 to 80 to 15 to 85),
   the aerosol container comprising a stem valve having a stem orifice with a diameter from 0.27 to 0.35 mm, and preferably additionally bearing a swirl nozzle.

A particularly preferred aerosol spray of this invention is an agent, packaged in an aerosol container, containing in a cosmetically acceptable carrier:
i) 1.0 wt % to 2.8 wt %, preferably from 1.5 wt % to 2.8 wt %, based on total weight of the cosmetic agent, of at least one copolymer (a) comprising at least one structural unit of formula (I), at least one structural unit of formula (II) and at least one structural unit of formula (III-1)

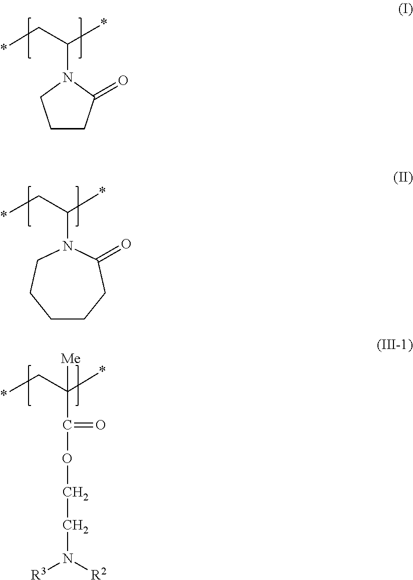

wherein R² and R³, mutually independently, are a (C₁ to C₄) alkyl group, and
ii) 0.4 wt % to 1.4 wt %, preferably from 0.6 wt % to 1.3 wt %, based on total weight of the cosmetic agent, of at least one copolymer (b) having at least one structural unit of formula (I), at least one structural unit of formula (II) and at least one structural unit of formula (IV-1)

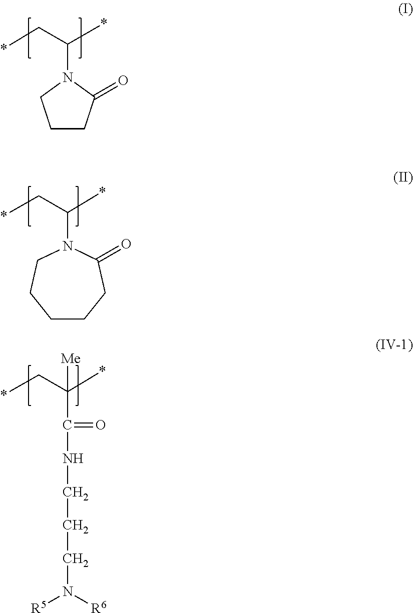

wherein R⁵ and R⁶, mutually independently, are a (C₁ to C₄) alkyl group, and
iii) at least one propellant (particularly as the only propellant, 30 to 55 wt % of a mixture of propane and butane at a weight ratio of propane to butane from 20 to 80 to 15 to 85),
   the aerosol container comprising a stem valve having a stem orifice with a diameter from 0.27 to 0.35 mm, and preferably additionally bearing a swirl nozzle, and
   wherein copolymer (a) and copolymer (b) are present in a weight ratio range (i) to (ii) from 1 to 1 to 1 to 5, particularly from 1 to 1.5 to 1 to 2.5.

Examples of (C₁ to C₄) alkyl groups according to formulae (III-1) and (IV-1) are methyl, ethyl, isopropyl, n-propyl, n-butyl, sec-butyl, isobutyl, and tert-butyl.

A very particularly preferred aerosol spray of this invention is an agent packaged in an aerosol container containing in a cosmetically acceptable carrier:
i) 1.0 wt % to 2.8 wt %, preferably from 1.5 wt % to 2.8 wt %, based on total weight of the cosmetic agent, of at least one terpolymer of N-vinylpyrrolidone, N-vinylcaprolactam, and N,N-dimethylaminoethyl methacrylate as copolymer (a), and
ii) 0.4 wt % to 1.4 wt %, preferably from 0.6 wt % to 1.3 wt %, based on total weight of the cosmetic agent, of at least one terpolymer of N-vinylpyrrolidone, N-vinylcaprolactam, and N,N-dimethylaminopropyl methacrylamide as copolymer (b), and
iii) at least one propellant (particularly as the only propellant, 30 to 55 wt % of a mixture of propane and butane at a weight ratio of propane to butane from 20 to 80 to 15 to 85),
   the aerosol container comprising a stem valve having a stem orifice with a diameter from 0.27 to 0.35 mm, and preferably additionally bearing a swirl nozzle, and
   wherein copolymer (a) and copolymer (b) are present in a weight ratio range (i) to (ii) from 1 to 1 to 1 to 5, particularly from 1 to 1.5 to 1 to 2.5.

All embodiments of aerosol sprays according to the present invention preferably possess a spray rate from 6.5 to 10.0 g/10 s.

Suitable propellants according to the present invention for generating aerosol foam include N₂O, dimethyl ether, CO₂, air, alkanes having 3 to 5 carbon atoms such as propane, n-butane, isobutane, n-pentane, and isopentane, and mixtures thereof. Dimethyl ether, propane, n-butane, isobutane, and mixtures thereof are preferred.

Use of the additional preferred ingredients recited above and of the utilization quantities or utilization quantity ratios identified as preferred (see above) is also preferred in the context of this/these embodiment(s).

In the embodiment as aerosol foam, propellants suitable according to the present invention include N₂O, dimethyl ether, CO₂, air, alkanes having 3 to 5 carbon atoms such as propane, n-butane, isobutane, n-pentane, and isopentane, and mixtures thereof. According to a preferred embodiment of aerosol foam, the aforesaid alkanes, mixtures of the aforesaid alkanes, or mixtures of the aforesaid alkanes with dimethyl ether are preferably used as the only propellant. The invention also expressly includes, however, the concurrent use of propellants of the chlorofluorocarbon type, particularly fluorocarbons.

For a given spray apparatus, the size of the foam bubbles and the respective size distribution can be adjusted by the quantitative ratio between the propellant and the other constituents of the preparations.

When conventional aerosol containers are used, aerosol foam products contain the propellant preferably in quantities from 1 to 35 wt %, based on total product. Quantities from 2 to 30 wt %, particularly from 3 to 15 wt %, are particularly preferred.

In order to foam gel-type agents in a two-chamber aerosol container, isopentane is preferably suitable as a propellant that is incorporated into the agents and is packaged in the first chamber of the two-chamber aerosol container. Packaged in the second chamber of the two-chamber aerosol container is at least one further propellant different from isopentane that builds up in the two-chamber aerosol container a higher pressure than the isopentane. The propellants of the second chamber are preferably selected from N₂O, dimethyl ether, CO₂, air, alkanes having 3 or 4 carbon atoms (such as propane, n-butane, isobutane), and mixtures thereof.

Use of additional preferred ingredients recited previously, and of the utilization quantities or utilization ratios identified as preferred (see above) is preferred in the context of this embodiment as well.

Agents according to the present invention and products that contain said agents, particularly aerosol hair sprays and aerosol hair foams, are notable in particular in that they impart a very natural shine and a strong hold to the hair that is treated.

A second subject of the invention is the use of an agent of the first subject of the invention to generate shine on keratin-containing fibers, particularly human hair.

Products according to the present invention containing these agents, particularly aerosol hair sprays, are notable in that they impart a very strong, lasting hairstyle hold to treated hair while the hair remains flexible.

A third subject of the invention is therefore the use of an agent of the first subject of the invention for temporary deformation of keratin-containing fibers, particularly human hair.

A fourth subject of the invention is a method for treating keratin-containing fibers, particularly human hair, in which, using a delivery apparatus, an agent in accordance with the first subject of the invention is applied as a spray onto the keratin-containing fibers.

It is preferred according to the present invention that a shape is imparted to the keratin-containing fibers, and that that shape is fixed in place by the agent of the first subject of the invention.

It is further preferred if, after application of the agent according to the present invention, the agent remains on the keratin-containing fibers (i.e., is not rinsed out again).

The delivery apparatuses or aerosol products recited previously are preferred according to the present invention.

A fifth subject of the invention is a method for treating keratin-containing fibers, particularly human hair, in which, using a delivery apparatus, an agent in accordance with the first subject of the invention is foamed into a foam and the resulting foam is applied onto the keratin-containing fibers.

It is preferred according to the present invention that a shape is imparted to the keratin-containing fibers, and that that shape is fixed in place by the agent of the first subject of the invention.

It is further preferred if, after application of the agent according to the present invention, the agent according to the present invention remains on the keratin-containing fibers (i.e., is not rinsed out again).

The delivery apparatuses (see above) recited previously are preferred according to the present invention.

The examples that follow are intended to explain the subject matter of the present invention without in any way limiting it.

### EXAMPLES

The quantitative indications below are understood, unless otherwise noted, as percentages by weight.

The following formulations were prepared by mixing the raw materials indicated, and decanted into an aerosol can having a Coster model KE valve and a Coster model V06 “standard micromist insert” swirl nozzle. The aerosol cans were sealed with the valve, and the corresponding propellant was then added:

| Raw material | A | B | C |
|---|---|---|---|
| Ethanol, denatured | 55.00 | 39.95 | 42.75 |
| Advantage LC-E ¹ | 6.50 | 6.50 | 4.50 |
| Aquaflex SF-40 ² | 3.00 | 3.00 | 2.00 |
| Neoheliopan E 1000 ³ | 0.10 | 0.10 | 0.20 |
| Triethyl citrate | 0.20 | 0.20 | — |
| Isopropyl myristate | — | — | 0.10 |
| Vitamin A acetate | — | — | 0.20 |
| Perfume | 0.25 | 0.25 | 0.25 |
| Butane/isobutane | 21.00 | — | — |
| Mixture of 15% propane, | 14.00 | 50.00 | 50.00 |
| 85% butane/isobutane | | | |

¹ INCI name: Vinylcaprolactam/VP/Dimethylaminoethyl Methacrylate Copolymer, Laurylpyrrolidone; 37 wt % active substance in ethanol with addition of N-laurylpyrrolidone (ISP)
² INCI name: VP/Vinylcaprolactam/VP/DMAPA Acrylates Copolymer, Alcohol Denat.; 38-42 wt % active substance in ethanol (ISP)
³ 4-Methoxycinnamic acid isoamyl ester (INCI name: Isoamyl-p-Methoxycinnamate) (Symrise)

All resulting aerosol sprays A, B, and C had a spray rate from 0.65 to 1.0 g/s. After application onto the hair, the agents according to the present invention produced an outstanding, flexible hairstyle hold. The hair attained a natural gloss.

We claim:

1. Cosmetic agent comprising, in a cosmetically acceptable carrier:
i) 1.0 wt % to 2.8 wt % of at least one copolymer (a) having at least one structural unit of formula (I), at least one structural unit of formula (II) and at least one structural unit of formula (III) 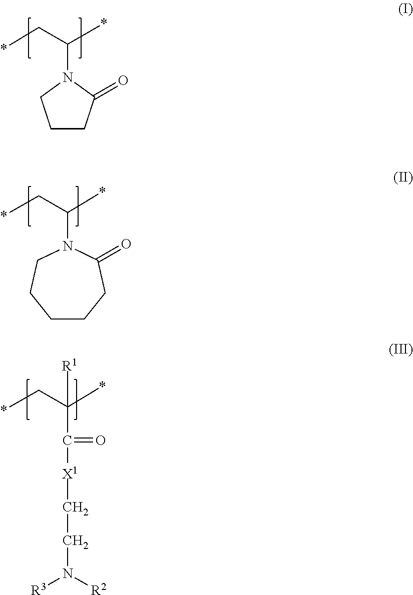
wherein
R¹ is a hydrogen atom or a methyl group,
X¹ is an oxygen atom or an NH group,
R² and R³, mutually independently, are a (C1 to C4) alkyl group, and
ii) 0.4 wt % to 1.4 wt % of at least one copolymer (b) having at least one structural unit of formula (I), at least one structural unit of formula (II) and at least one structural unit of formula (IV) 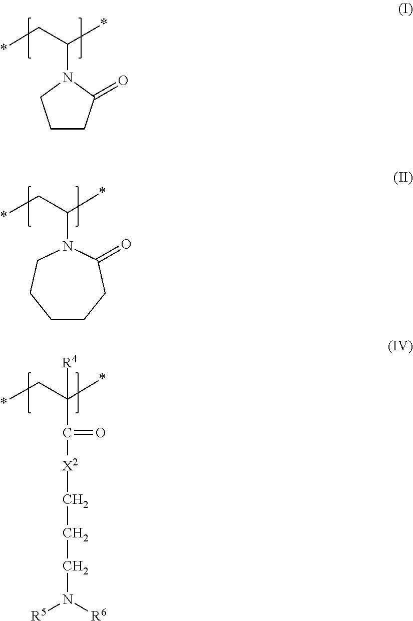
wherein
R⁴ is a hydrogen atom or a methyl group,
X² is an oxygen atom or an NH group,
R⁵ and R⁶, mutually independently, are a (C1 to C4) alkyl group.

2. Agent according to claim 1, wherein R¹ is a methyl group.

3. Agent according to claim 1, wherein X¹ is an oxygen atom.

4. Agent according to claim 1, wherein R² and R³ are a methyl group.

5. Agent according to claim 1, wherein R⁴ is a methyl group.

6. Agent according to claim 1, wherein X² is an NH group.

7. Agent according to claim 1, wherein R⁵ and R⁶ are a methyl group.

8. Agent according to claim 1, wherein copolymer (a) and copolymer (b) are present in a weight ratio range (i) to (ii) from 1 to 1 to 1 to 5.

9. Agent according to claim 1 further comprising at least one alcohol having 2 to 6 carbon atoms and 1 to 3 hydroxyl groups.

10. Agent according to claim 9, wherein the alcohol is chosen from ethanol, isopropanol, 1,2-propylene glycol, glycerol, n-butanol, 1,3-butylene glycol and mixtures thereof.

11. Agent according to claim 1 further comprising at least one compound of formula (E) 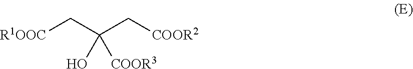
wherein R¹, R², and R³, mutually independently, are a (C₂ to C₆) alkyl group.

12. Agent according to claim 1 further comprising at least one propellant.

13. Agent according to claim 1, wherein the agent is present as aerosol spray or as aerosol foam.

14. Method of generating gloss on keratin-containing fibers comprising applying an agent according to claim 1 onto keratin-containing fibers.

15. Method for treating keratin-containing fibers comprising applying as a spray using a delivery apparatus an agent according to claim 1 onto the keratin-containing fibers.

16. Method for treating keratin-containing fibers comprising foaming using a delivery apparatus an agent according to claim 1 into a foam, and applying the resulting foam onto the keratin-containing fibers.
